SEQUENCE LISTING (10 sequences)

SEQ ID NO: 1 - protein, 808 aa, synthetic construct
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLDFLRLHSHQGKNLMLSEKIQNLNTLQHTLRKAEEYLAELKSETLYEEFEAKFEEIGLERGWGDNAERVLDMIRLLLDLLEAPDPSTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENDEHLCVLKDKKKPILFTMARLDRVKNLSGLVEWYGKNTRLRELVNLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADLLADFFTKCKEDPSHWDEISKGGLQRIEEKYTWQIYSQRLLTLTGVYGFWKHVSNLDRLEHRRYLEMFYALKYRPLAQAVPLAQDD

SEQ ID NO: 2 - protein, 460 aa, synthetic construct
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 3 - protein, 481 aa, synthetic construct
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 4 - protein, 442 aa, synthetic construct
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQGIPAGKLLVSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKPENSTVFVSFGSEYFLSKEDMEEIAFALEASNVNFIWVVRFPKGEERNLEDALPEGFLERIGERGRVLDKFAPQPRILNHPSTGGFISHCGWNSVMESIDFGVPIIAMPIHNDQPINAKLMVELGVAVEIVRDDDGKIHRGEIAEALKSVVTGETGEILRAKVREISKNLKSIRDEEMDAVAEELIQLCRNSNKSK

SEQ ID NO: 5 - protein, 458 aa, synthetic construct
MENKTETTVRRRRRIILFPVPFQGHINPILQLANVLYSKGFAITILHTNFNKPKTSNYPHFTFRFILDNDPQDERISNLPTHGPLAGMRIPIINEHGADELRRELELLMLASEEDEEVSCLITDALWYFAQDVADSLNLRRLVLMTSSLFNFHAHVSLPQFDELGYLDPDDKTRLEEQASGFPMLKVKDIKSAYSNWQIGKEILGKMIKQTKASSGVIWNSFKELEESELETVIREIPAPSFLIPLPKHLTASSSSLLDHDRTVFEWLDQQAPSSVLYVSFGSTSEVDEKDFLEIARGLVDSGQSFLWVVRPGFVKGSTWVEPLPDGFLGERGKIVKWVPQQEVLAHPAIGAFWTHSGWNSTLESVCEGVPMIFSSFGGDQPLNARYMSDVLRVGVYLENGWERGEVVNAIRRVMVDEEGEYIRQNARVLKQKADVSLMKGGSSYESLESLVSYISSL

SEQ ID NO: 6 - protein, 462 aa, synthetic construct
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 7 - protein, 463 aa, synthetic construct
MAGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTK

SEQ ID NO: 8 - protein, 463 aa, synthetic construct
MDGDGNSSSSSSPLHVVICPWLALGHLLPCLDIAERLASRGHRVSFVSTPRNIARLPPLRPAVAPLVEFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 9 - protein, 473 aa, synthetic construct
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

SEQ ID NO: 10 - protein, 464 aa, synthetic construct
MENGSSPLHVVIFPWLAFGHLLPFLDLAERLAARGHRVSFVSTPRNLARLRPVRPALRGLVDLVALPLPRVDGLPDGAEATSDVPFEKFELHRKAFDGLAAPFSAFLDAACAGDKRPDWVIPDFMHYWVAAAAQKRGVPCAVLIPCSADVMALYGQPTETSTEQPEAIARSMAAEAPSFEAERNTEEYGTAGASGVSIMTRFSLTLKWSKLVALRSCPELEPGVFTTLTRVYSKPVVPFGLLPPRRDGAHGVRKNGEDDGAIIRWLDEQPAKSVVYVALGSEAPVSADLLRELAHGLELAGTRFLWALRRPAGVNDGDSILPNGFLERTGERGLVTTGWVPQVSILAHAAVCAFLTHCGWGSVVEGLQFGHPLIMLPIIGDQGPNARFLEGRKVGVAVPRNHADGSFDRSGVAGAVRAVAVEEEGKAFAANARKLQEIVADRERDERCTDGFIHHLTSWNELEA

(19) United States
(12) Patent Application Publication
(10) Pub. No.: US 2026/0009063 A1
(43) Pub. Date: Jan. 8, 2026

(54) HIGH-PURITY STEVIOL GLYCOSIDES

(71) Applicant: PURECIRCLE SDN. BHD., NEGERI SEMBILAN (MY)

(72) Inventors: Avetik MARKOSYAN, Negeri Semilan (MY); Pei Chen KOH, Negeri Sembilan (MY); Mohamad Afzaal BIN HASIM, Negeri Sembilan (MY); Khairul NIZAM BIN NAWI, Negeri Sembilan (MY); Saravanan A/L RAMANADACH, Negeri Sembilan (MY); Kristina CHKHAN, Negeri Sembilan (MY)

(21) Appl. No.: 18/881,823

(22) PCT Filed: Jun. 30, 2023
(86) PCT No.: PCT/MY2023/050051

§ 371 Date: Jan. 7, 2025

(30) Foreign Application Priority Data
Jul. 7, 2022 (MY) PI2022003636
Feb. 17, 2023 (MY) PI2023000838

(51) Int. Cl. C12P 19/56; A23L 27/30; C12N 1/14; C12N 1/20; C12N 9/10; C12R 1/01; C12R 1/645

(52) U.S. Cl. CPC C12P 19/56; A23L 27/30; C12N 1/14; C12N 1/20; C12N 9/1051; C12Y 204/01242; C12R 2001/01; C12R 2001/645

(57) ABSTRACT

Methods of preparing highly purified steviol glycosides are described. The methods include utilizing enzyme preparations and recombinant microorganisms for converting various starting compositions to target steviol glycosides. The highly purified steviol glycosides are useful as non-caloric sweetener, flavor enhancer, sweetness enhancer, flavor stabilizer, flavoring with modifying properties (FMP), foaming suppressor and solubility enhancing agent in consumable products such as any food, beverages, pharmaceutical compositions, tobacco products, nutraceutical compositions, and oral hygiene compositions.

### TECHNICAL FIELD

The present invention relates to a process for preparing compositions comprising steviol glycosides, including highly purified steviol glycoside compositions.

### BACKGROUND OF THE INVENTION

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling for their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

Although methods are known for preparing steviol glycosides from Stevia rebaudiana, many of these methods are unsuitable for use commercially.

Accordingly, there remains a need for simple, efficient, and economical methods for preparing compositions comprising steviol glycosides, including highly purified steviol glycoside compositions.

### SUMMARY OF THE INVENTION

As used herein, the abbreviation term “reb” refers to “rebaudioside”. Both terms have the same meaning and may be used interchangeably.

As used herein, “biocatalysis” or “biocatalytic” refers to the use of natural or genetically engineered biocatalysts, such as enzymes, or cells comprising one or more enzyme, capable of single or multiple step chemical transformations on organic compounds.

Biocatalysis processes include fermentation, biosynthesis, bioconversion and biotransformation processes. Both isolated enzymes, and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7, rebaudioside N8, naturally occurring steviol glycosides, synthetic steviol glycosides, e.g. enzymatically glucosylated steviol glycosides and combinations thereof.

The present invention provides a process for preparing a composition comprising a target steviol glycoside by contacting a starting composition comprising an organic substrate with a microbial cell and/or enzyme preparation, thereby producing a composition comprising a target steviol glycoside.

The starting composition can be any organic compound comprising at least one carbon atom. In one embodiment, the starting composition is selected from the group consisting of steviol glycosides, polyols or sugar alcohols, various carbohydrates.

The target steviol glycoside can be any steviol glycoside. In one embodiment, the target steviol glycoside is steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7, rebaudioside N8 or other synthetic steviol glycoside.

In one embodiment, the target steviol glycoside is stevioside M.

In one embodiment, the target steviol glycoside is rebaudioside C7.

In one embodiment, the target steviol glycoside is rebaudioside C8.

In one embodiment, the target steviol glycoside is rebaudioside E13.

In one embodiment, the target steviol glycoside is rebaudioside E14.

In one embodiment, the target steviol glycoside is rebaudioside D13.

In one embodiment, the target steviol glycoside is rebaudioside D14.

In one embodiment, the target steviol glycoside is rebaudioside H7.

In one embodiment, the target steviol glycoside is rebaudioside H8.

In one embodiment, the target steviol glycoside is rebaudioside N7.

In one embodiment, the target steviol glycoside is rebaudioside N8.

In some preferred embodiments, enzyme preparation comprising one or more enzymes, or a microbial cell comprising one or more enzymes, capable of converting the starting composition to target steviol glycosides are used. The enzyme can be located on the surface and/or inside the cell. The enzyme preparation can be provided in the form of a whole cell suspension, a crude lysate or as purified enzyme(s). The enzyme preparation can be in free form or immobilized to a solid support made from inorganic or organic materials.

In some embodiments, a microbial cell comprises the necessary enzymes and genes encoding thereof for converting the starting composition to target steviol glycosides. Accordingly, the present invention also provides a process for preparing a composition comprising a target steviol glycoside by contacting a starting composition comprising an organic substrate with a microbial cell comprising at least one enzyme capable of converting the starting composition to target steviol glycosides, thereby producing a medium comprising at least one target steviol glycoside.

In one embodiment, the enzymes necessary for converting the starting composition to target steviol glycosides include the NDP-glucosyltransferases (NGTs), ADP-glucosyltransferases (AGTs), CDP-glucosyltransferases (CGTs), GDP-glucosyltransferases (GGTs), TDP-glucosyltransferases (TGTs), UDP-glucosyltransferases (UGTs). Optionally it may include NDP-recycling enzyme, ADP-recycling enzyme, CDP-recycling enzyme, GDP-recycling enzyme, TDP-recycling enzyme, and/or UDP-recycling enzyme.

In yet another embodiment, the enzymes necessary for converting the starting composition to target steviol glycosides include the NDP-rhamnosyltransferases (NRhaTs), ADP-rhamnosyltransferases (ARhaTs), CDP-rhamnosyltransferases (CRhaTs), GDP-rhamnosyltransferases (GRhaTs), TDP-rhamnosyltransferases (TRhaTs), UDP-rhamnosyltransferases (URhaTs). Optionally it may include NDP-recycling enzyme, ADP-recycling enzyme, CDP-recycling enzyme, GDP-recycling enzyme, TDP-recycling enzyme, and/or UDP-recycling enzyme.

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group consisting of geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

The UDP-rhamnosyltransferase can be any UDP-rhamnosyltransferase capable of adding at least one rhamnose unit to steviol and/or a steviol glycoside substrate to provide the target steviol glycoside.

As used hereinafter, the term “SuSy_AT”, unless specified otherwise, refers to sucrose synthase having amino-acid sequence SEQ ID 1, or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 1 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 1 polypeptide. Sucrose synthase produces UDP-glucose by transferring glucose from a glucose donor, e.g. sucrose to UDP. UDP-glucose is then used by glucosyltransferase for transferring the glucose to a steviol-containing compound to produce a target compound.

As used hereinafter, the term “UGT74G1”, unless specified otherwise, refers to UDP-glucosyltransferase having amino-acid sequence SEQ ID 2 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 2 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 2 polypeptide.

As used hereinafter, the term “UGT85C2”, unless specified otherwise, refers to UDP-glucosyltransferase having amino-acid sequence SEQ ID 3 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 3 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 3 polypeptide.

As used hereinafter, the term “UGTS12”, unless specified otherwise, refers to UDP-glucosyltransferase having amino-acid sequence SEQ ID 4 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 4 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 4 polypeptide.

As used hereinafter, the term “UGT76G1”, unless specified otherwise, refers to UDP-glucosyltransferase having amino-acid sequence SEQ ID 5 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 5 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 5 polypeptide.

As used hereinafter, the term “URhaT12V1”, unless specified otherwise, refers to UDP-rhamnosyltransferase having amino-acid sequence SEQ ID 6 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 6 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 6 polypeptide.

As used hereinafter, the term “URhaT22V1”, unless specified otherwise, refers to UDP-rhamnosyltransferase having amino-acid sequence SEQ ID 7 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 7 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 7 polypeptide.

As used hereinafter, the term “URhaT32V1”, unless specified otherwise, refers to UDP-rhamnosyltransferase having amino-acid sequence SEQ ID 8 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 8 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 8 polypeptide.

As used hereinafter, the term “URhaT42V1”, unless specified otherwise, refers to UDP-rhamnosyltransferase having amino-acid sequence SEQ ID 9 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 9 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 9 polypeptide.

As used hereinafter, the term “URhaT52V1”, unless specified otherwise, refers to UDP-rhamnosyltransferase having amino-acid sequence SEQ ID 10 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 10 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 10 polypeptide.

In one embodiment, steviol biosynthesis enzymes, UDP-glucosyltransferases and UDP-rhamnosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases and UDP-rhamnosyltransferases are synthesized.

In one embodiment, the UDP-glucosyltransferase is selected from group including UGT74G1, UGT85C2, UGTS12, UGT76G1 and UGTs having substantial (>85%, >86%, >87%, >88%, >89%, >90%, >91%, >92%, >93%, >94%, >95%, >96%, >97%, >98%, >99%) amino-acid sequence identity to SEQ ID 2, SEQ ID 3, SEQ ID 4 and SEQ ID 5, respectively as well as isolated nucleic acid molecules that code for these UGTs. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to these polypeptides.

In one embodiment, the UDP-rhamnosyltransferase is selected from group including URhaT12V1, URhaT22V1, URhaT32V1, URhaT42V1, URhaT52V1 and URhaTs having substantial (>85%, >86%, >87%, >88%, >89%, >90%, >91%, >92%, >93%, >94%, >95%, >96%, >97%, >98%, >99%) amino-acid sequence identity to SEQ ID 6, SEQ ID 7, SEQ ID 8, SEQ ID 9 and SEQ ID 10, respectively as well as isolated nucleic acid molecules that code for these URhaTs. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to these polypeptides.

In one embodiment, steviol biosynthesis enzymes, UGTs, URhaTs, UDP-glucose recycling system, UDP-rhamnose recycling system and UDP-rhamnose synthesis system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with SEQ ID 2.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In another embodiment, the UDP-rhamnosyltransferase is any UDP-rhamnosyltransferase capable of adding at least one rhamnose unit to the existing glucose at C13 of any starting steviol glycoside to give a target steviol glycoside with at least one additional rhamnose bearing at least one alpha 1→2 rhamnopyranoside glycosidic linkage(s) at the newly formed glycosidic bond(s). In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with SEQ ID 2.

In another embodiment, the UDP-rhamnosyltransferase is any UDP-rhamnosyltransferase capable of adding at least one rhamnose unit to steviolmonoside to form steviolbioside C. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to steviolmonoside to form steviolbioside D. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolmonoside A to form steviolbioside G. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolbioside C to form dulcoside C. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolbioside C to form dulcoside A. In a particular embodiment, the UDP-glucosyltransferase is UGT74G1 or a UGT having >85% amino-acid sequence identity with SEQ ID 2.

In another embodiment, the UDP-rhamnosyltransferase is any UDP-rhamnosyltransferase capable of adding at least one rhamnose unit to steviolbioside D to form dulcoside C. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to steviolbioside D to form rebaudioside G. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolbioside D to form stevioside M. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rubusoside to form dulcoside A. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rubusoside to form rebaudioside G. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside A. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rubusoside to form stevioside L. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside A to form stevioside K. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolbioside G to form stevioside L. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside G to form stevioside K. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to dulcoside C to form rebaudioside C. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to dulcoside C to form rebaudioside C7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to dulcoside A to form rebaudioside C. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to dulcoside A to form rebaudioside C5. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to dulcoside A to form rebaudioside C8. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside G to form rebaudioside C. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside G to form rebaudioside E13. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside G to form rebaudioside E14. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside G to form rebaudioside E4. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to stevioside M to form rebaudioside C7. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to stevioside M to form rebaudioside E13. In a particular embodiment, the UDP-glucosyltransferase is UGT74G1 or a UGT having >85% amino-acid sequence identity with SEQ ID 2.

In another embodiment, the UDP-rhamnosyltransferase is any UDP-rhamnosyltransferase capable of adding at least one rhamnose unit to stevioside A to form rebaudioside C5. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to stevioside A to form rebaudioside E4. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E12. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to stevioside L to form rebaudioside C8. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to stevioside L to form rebaudioside E14. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside L to form rebaudioside E12. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to stevioside K to form rebaudioside E12. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to rebaudioside C to form rebaudioside H7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C to form rebaudioside K. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C to form rebaudioside K2. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C7 to form rebaudioside H7. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to rebaudioside C5 to form rebaudioside K. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside C5 to form rebaudioside H8. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C8 to form rebaudioside K2. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside C8 to form rebaudioside H8. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside E13 to form rebaudioside H7. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10. In another embodiment, the UDP-glucosyltransferase is any UDP-glucosyltransferase capable of adding at least one glucose unit to rebaudioside E13 to form rebaudioside D13. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside E4 to form rebaudioside K. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside E4 to form rebaudioside D13. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside E4 to form rebaudioside D14. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside E12 to form rebaudioside H8. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside E12 to form rebaudioside D14. In a particular embodiment, the UDP-glucosyltransferase is UGT76G1, or a UGT having >85% amino-acid sequence identity with SEQ ID 5.

In another embodiment, the UDP-rhamnosyltransferase is any UDP-rhamnosyltransferase capable of adding at least one rhamnose unit to rebaudioside E14 to form rebaudioside K2. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside E14 to form rebaudioside D14. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside H7 to form rebaudioside N7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside H8 to form rebaudioside N8. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside K to form rebaudioside N7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside K to form rebaudioside N8. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside K2 to form rebaudioside N8. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside D13 to form rebaudioside N7. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-rhamnosyltransferase is any UDP-rhamnosyltransferase capable of adding at least one rhamnose unit to rebaudioside D14 to form rebaudioside N8. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside C to form rebaudioside N7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C to form rebaudioside N8. In a particular embodiment, the UDP-glucosyltransferase is UGTS12 or a UGT having >85% amino-acid sequence identity with SEQ ID 4.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose. The UDP recycling enzyme can be sucrose synthase SuSy_At or a sucrose synthase having >85% amino-acid sequence identity with SuSy_At and the recycling substrate can be sucrose.

Optionally, the method of the present invention further comprises recycling UDP to provide UDP-rhamnose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-rhamnosyltransferase and UDP-rhamnose. The recycling substrate can be rhamnosyl-containing residue.

In one embodiment, UDP-rhamnose can be synthesized from UDP-glucose by UDP-rhamnose synthase system. In another embodiment, UDP-rhamnose can be synthesized from UDP-glucose by trifunctional UDP-rhamnose synthase NRF1 or NR32, as it is described in WO2020/205685A1, the content of which is hereby incorporated by reference, or a UDP-rhamnose synthase having >85% amino-acid sequence identity with NRF1 or NR32.

In one embodiment, the UDP-glucose recycling catalyst is sucrose synthase SuSy_At or a sucrose synthase having >85% amino-acid sequence identity with SuSy_At.

In one embodiment, the recycling substrate for UDP-glucose recycling catalyst is sucrose.

In one embodiment, the UDP-rhamnose synthesis and recycling catalyst is trifunctional UDP-rhamnose synthase NRF1 or NR32 or a UDP-rhamnose synthase having >85% amino-acid sequence identity with NRF1 or NR32. Exemplary synthesis and recycling pathway and gene expression are provided in WO2020/205685A1 and WO2018/190378A1 respectively, the content of which is hereby incorporated by reference.

In one embodiment, the recycling substrate for UDP-rhamnose recycling catalyst is a molecule comprising rhamnosyl residue. In another embodiment, the synthesis substrate for UDP-rhamnose synthesis catalyst is UDP-glucose. Exemplary synthesis and recycling pathway and gene expression are provided in WO2020/205685A1 and WO2018/190378A1 respectively, the content of which is hereby incorporated by reference.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside C, rebaudioside H7, rebaudioside K, rebaudioside K2, rebaudioside N7 and/or rebaudioside N8.

Optionally, the method of the present invention further comprises separating the target steviol glycoside from the medium to provide a highly purified target steviol glycoside composition. The target steviol glycoside can be separated by at least one suitable method, such as, for example, crystallization, separation by membranes, centrifugation, extraction, chromatographic separation or a combination of such methods.

In one embodiment, the target steviol glycoside can be produced by the enzyme. In another embodiment, the target steviol glycoside is produced by enzymatic conversion. In one another embodiment, the converted steviol glycoside can be continuously removed from the medium. In yet another embodiment, the target steviol glycoside is separated after the completion of the conversion reaction.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the conversion reaction.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 90% by weight of the target steviol glycoside. In particular embodiments, the composition comprises greater than about 95% by weight of the target steviol glycoside. In other embodiments, the composition comprises greater than about 99% by weight of the target steviol glycoside.

Unless otherwise indicated, weight percentages presented herein (e.g. percent by weight) are by weight of the total composition.

The target steviol glycoside can be in any polymorphic or amorphous form, including hydrates, solvates, anhydrous or combinations thereof.

Purified target steviol glycosides can be used in consumable products as a sweetener, flavor stabilizer, flavoring with modifying properties (FMP), foaming suppressor and/or solubility enhancing agent. Suitable consumer products include, but are not limited to, food, beverages, pharmaceutical compositions, tobacco products, nutraceutical compositions, oral hygiene compositions, and cosmetic compositions.

### BRIEF DESCRIPTION OF THE DRAWINGS

FIG. 1a shows the chemical structure of Steviolbioside C.

FIG. 1b shows the chemical structure of Steviolbioside D.

FIG. 1c shows the chemical structure of SteviolbiosideA.

FIG. 1d shows the chemical structure of Steviolbioside G.

FIG. 1e shows the chemical structure of Dulcoside C.

FIG. 1f shows the chemical structure of Stevioside M.

FIG. 1g shows the chemical structure of Stevioside A.

FIG. 1h shows the chemical structure of Stevioside K.

FIG. 1i shows the chemical structure of Stevioside L.

FIG. 1j shows the chemical structure of Rebaudioside C7.

FIG. 1k shows the chemical structure of Rebaudioside C5.

FIG. 1l shows the chemical structure of Rebaudioside C8.

FIG. 1m shows the chemical structure of Rebaudioside E13.

FIG. 1n shows the chemical structure of Rebaudioside E4.

FIG. 1o shows the chemical structure of Rebaudioside E12.

FIG. 1p shows the chemical structure of Rebaudioside E14.

FIG. 1q shows the chemical structure of Rebaudioside H7.

FIG. 1r shows the chemical structure of Rebaudioside H8.

FIG. 1s shows the chemical structure of Rebaudioside K2.

FIG. 1t shows the chemical structure of Rebaudioside D13.

FIG. 1u shows the chemical structure of Rebaudioside D14.

FIG. 2a-2e show the pathways of producing rebaudioside N7 and various steviol glycosides from steviol.

FIG. 3a-3e show the pathways of producing rebaudioside N8 and various steviol glycosides from steviol.

FIG. 4a shows the biocatalytic production of rebaudioside H7 from rebaudioside C using the enzyme UGTS12 and concomitant recycling of UDP to UDP-glucose via sucrose synthase SuSy_At.

FIG. 4b shows the biocatalytic production of rebaudioside K from rebaudioside C using the enzyme UGTS12 and concomitant recycling of UDP to UDP-glucose via sucrose synthase SuSy_At.

FIG. 4c shows the biocatalytic production of rebaudioside K2 from rebaudioside C using the enzyme UGTS12 and concomitant recycling of UDP to UDP-glucose via sucrose synthase SuSy_At.

FIG. 4d shows the biocatalytic production of rebaudioside N7 from rebaudioside C using the enzyme UGTS12 and concomitant recycling of UDP to UDP-glucose via sucrose synthase SuSy_At.

FIG. 4e shows the biocatalytic production of rebaudioside N8 from rebaudioside C using the enzyme UGTS12 and concomitant recycling of UDP to UDP-glucose via sucrose synthase SuSy_At.

FIG. 5a shows the biocatalytic production of rebaudioside N7 from rebaudioside H7 using the enzyme UGTS12 and concomitant recycling of UDP to UDP-glucose via sucrose synthase SuSy_At.

FIG. 5b shows the biocatalytic production of rebaudioside N7 from rebaudioside K using the enzyme UGTS12 and concomitant recycling of UDP to UDP-glucose via sucrose synthase SuSy_At.

FIG. 5c shows the biocatalytic production of rebaudioside N8 from rebaudioside K using the enzyme UGTS12 and concomitant recycling of UDP to UDP-glucose via sucrose synthase SuSyAt.

FIG. 5d shows the biocatalytic production of rebaudioside N8 from rebaudioside K2 using the enzyme UGTS12 and concomitant recycling of UDP to UDP-glucose via sucrose synthase SuSy_At.

FIG. 6a shows the HPLC chromatogram of rebaudioside C. The peak with retention time of 24.535 minutes corresponds to rebaudioside C.

FIG. 6b shows the HPLC chromatogram of the product of the biocatalytic production of rebaudioside N7 and rebaudioside N8 from rebaudioside C after 48 hours reaction. The peak at 6.743 minutes corresponds to rebaudioside N8. The peak at 8.678 minutes corresponds to rebaudioside N7. The peak at 11.410 minutes corresponds to rebaudioside K.

FIG. 6c shows the HPLC chromatogram of rebaudioside N7 after purification by HPLC. The peak with retention time of 7.968 minutes corresponds to rebaudioside N7.

FIG. 6d shows the HPLC chromatogram of rebaudioside N8 after purification by HPLC. The peak with retention time of 6.319 minutes corresponds to rebaudioside N8.

FIG. 7a shows the MSD chromatogram of rebaudioside N7.

FIG. 7b shows the mass spectrum of rebaudioside N7.

FIG. 8a shows the MSD chromatogram of rebaudioside N8.

FIG. 8b shows the mass spectrum of rebaudioside N8.

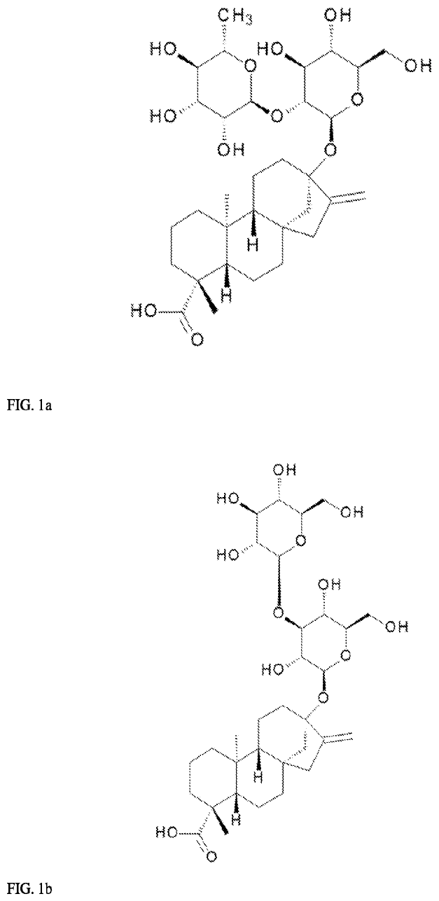

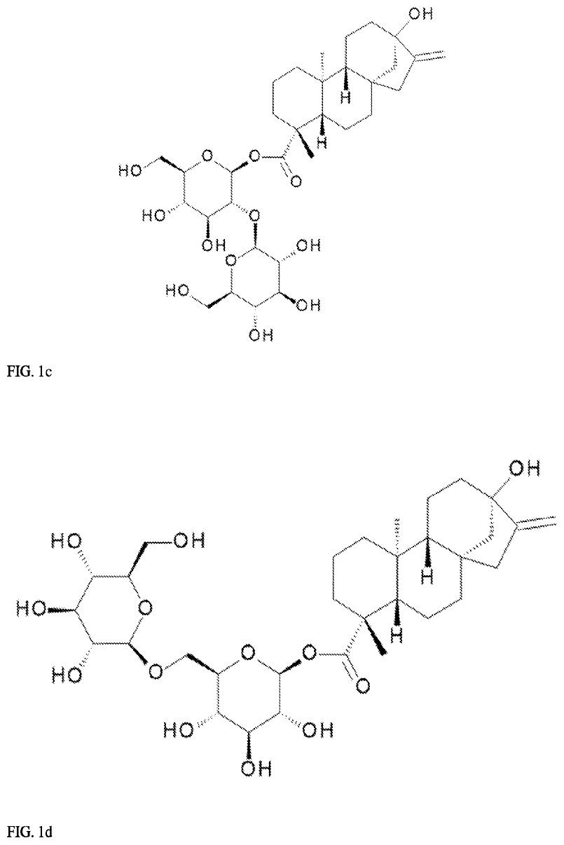

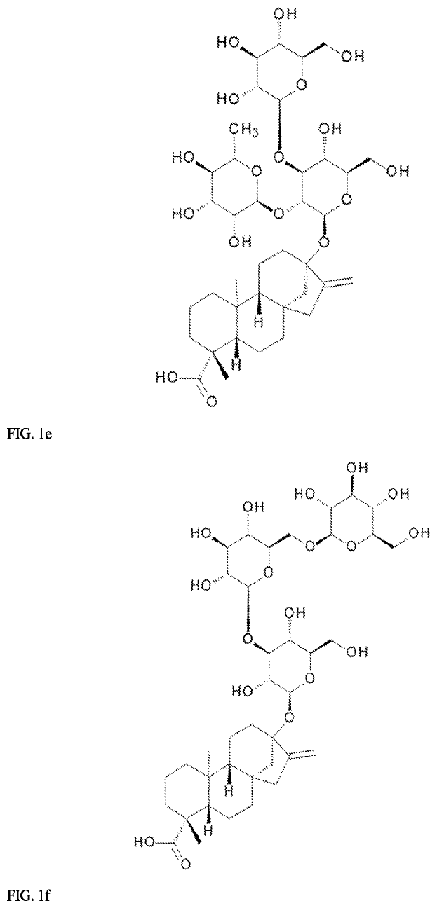

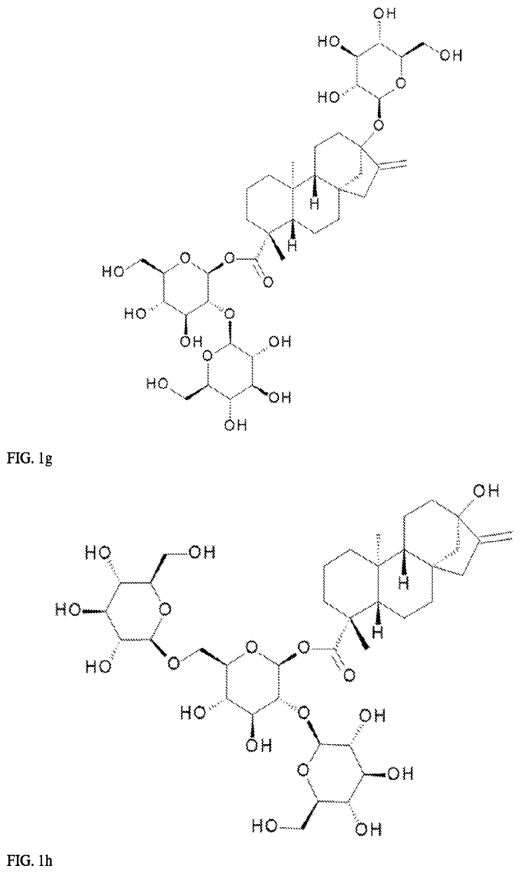

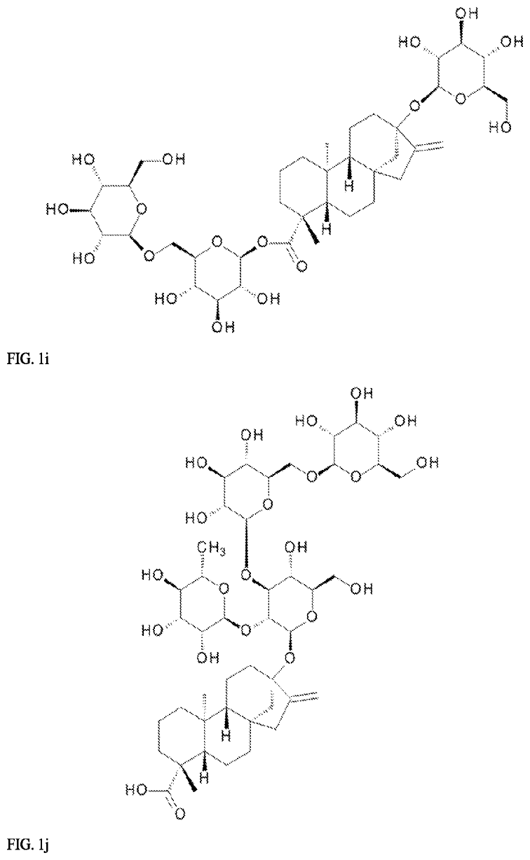

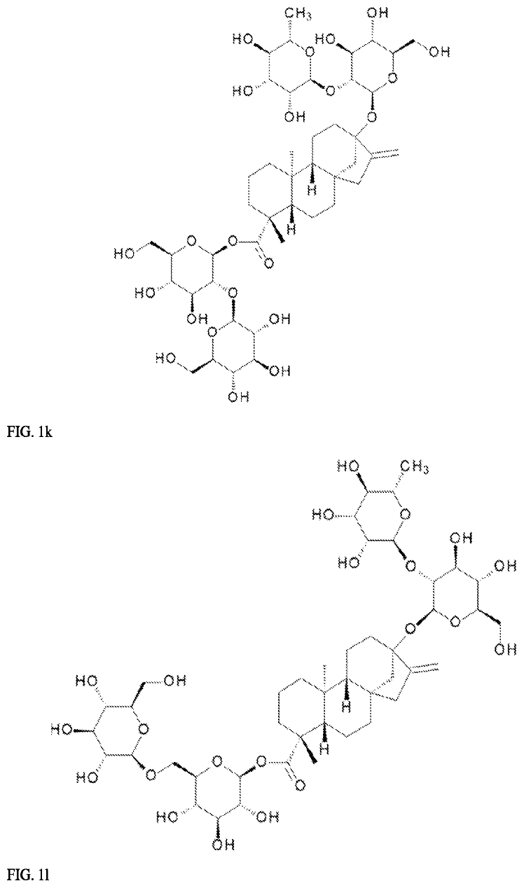

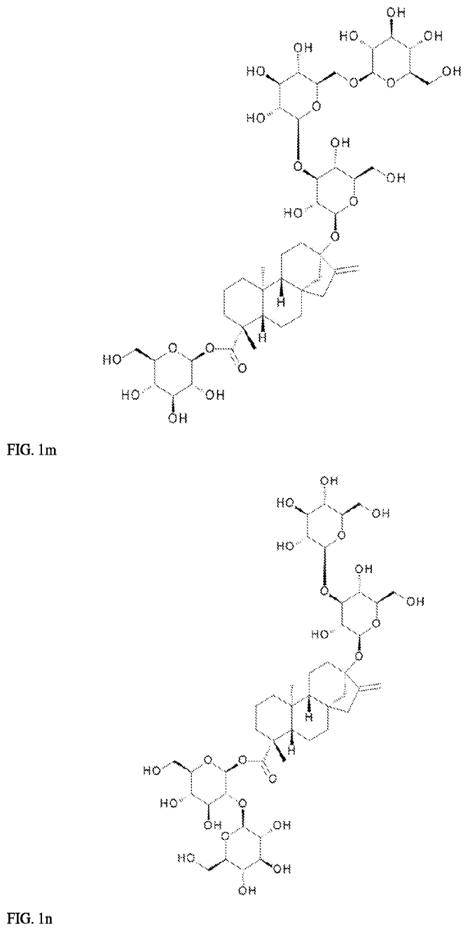

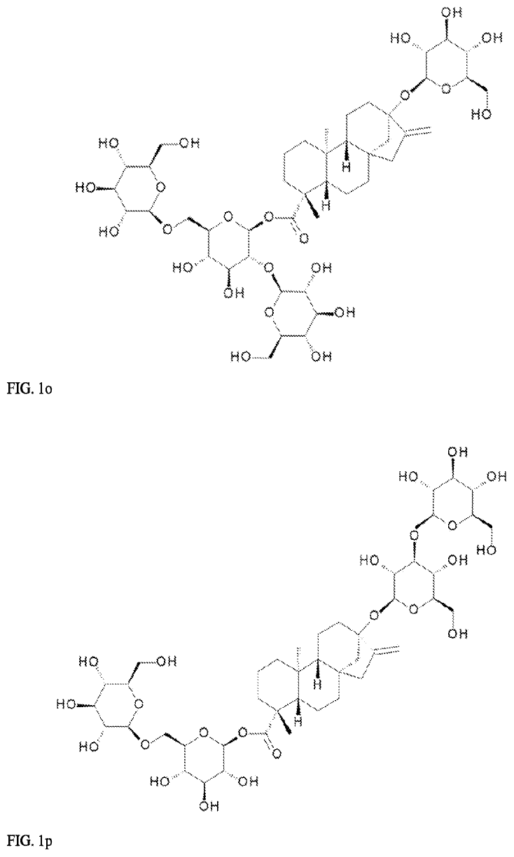

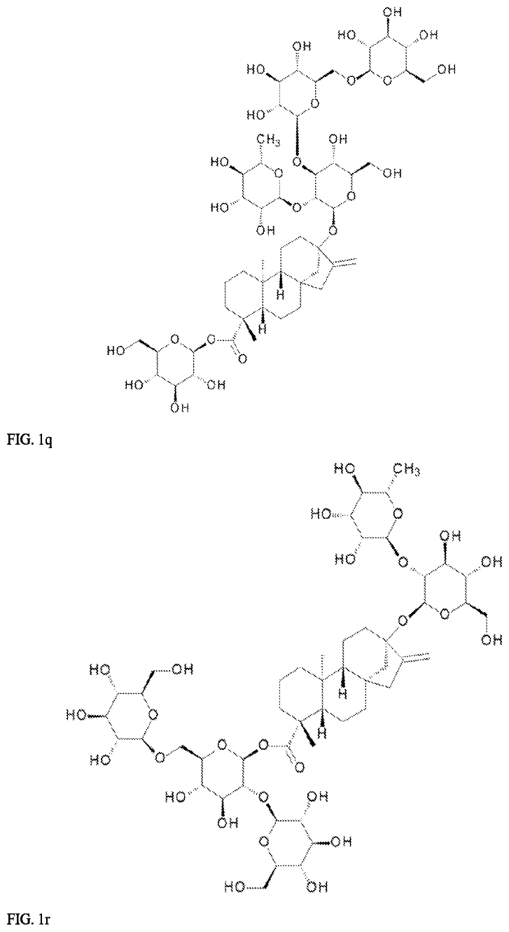

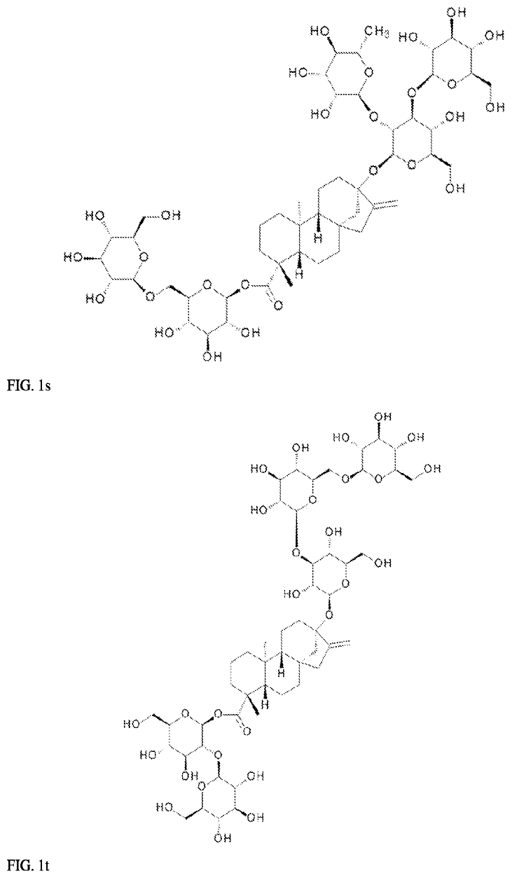

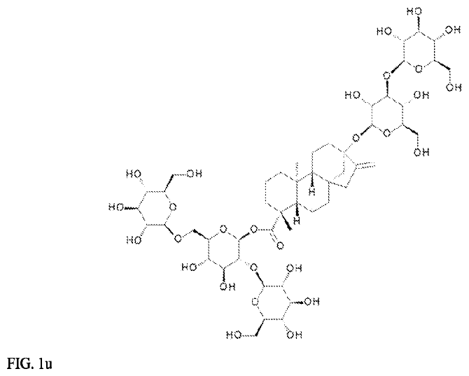

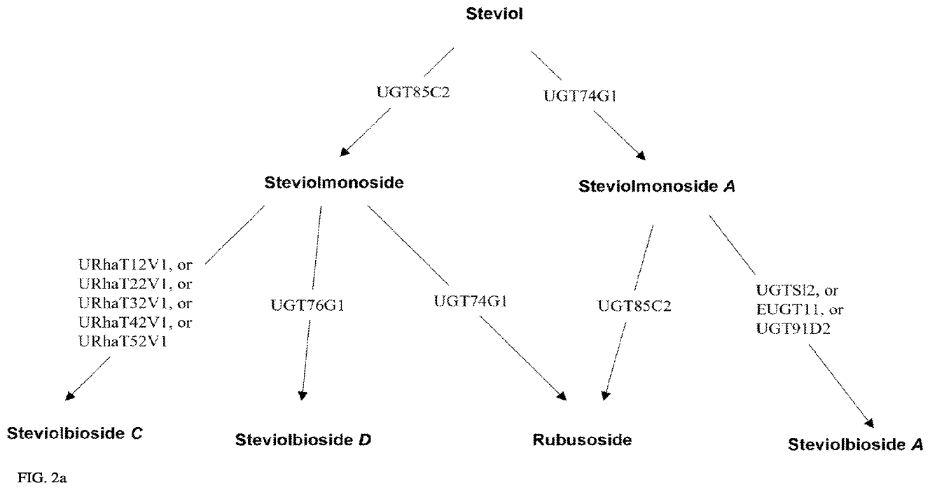

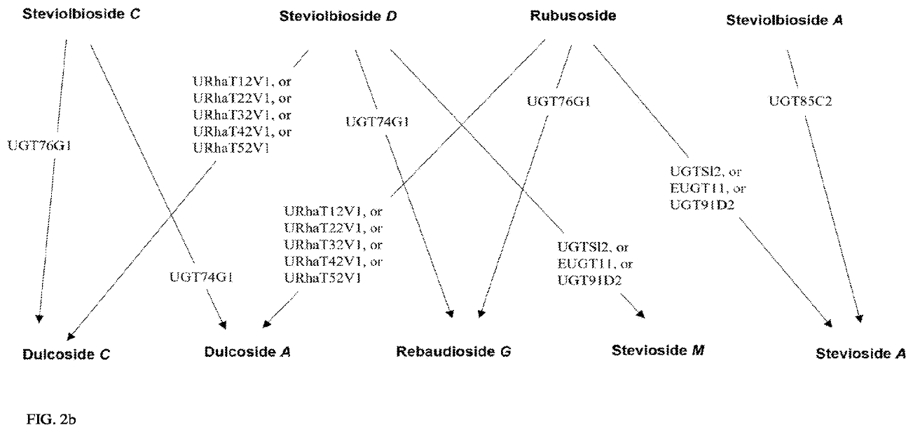

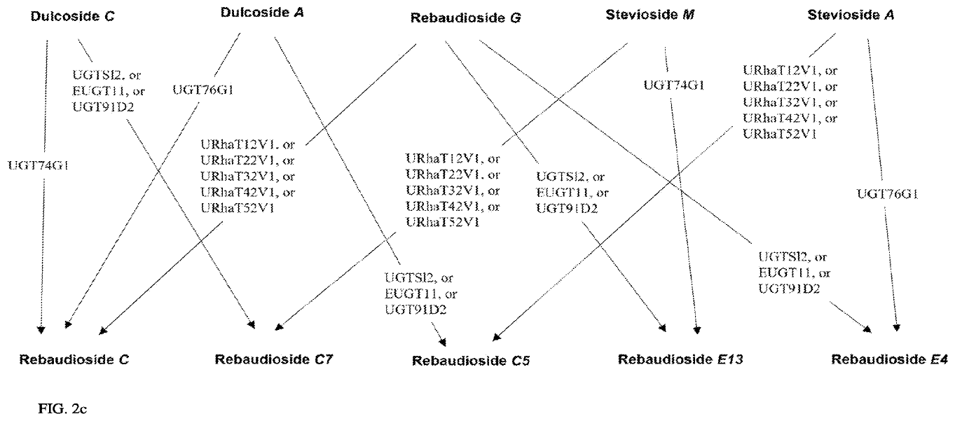

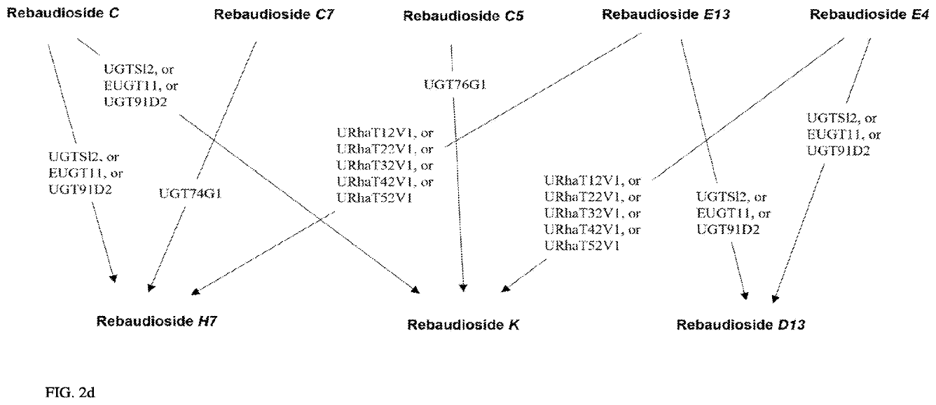

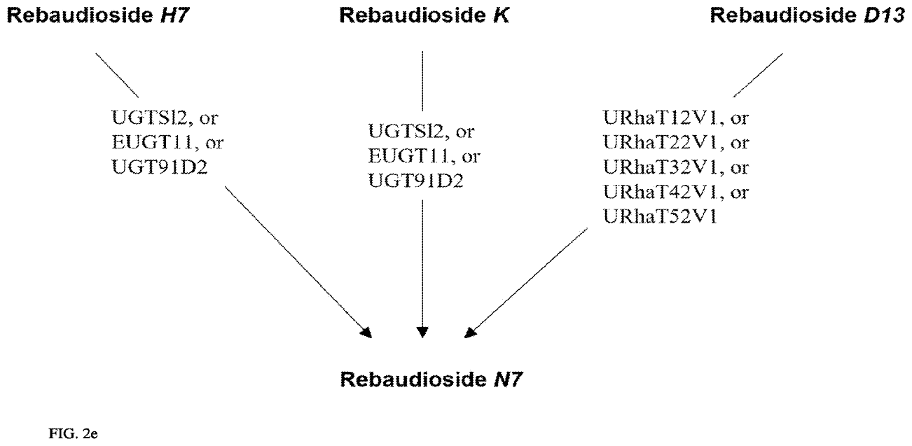

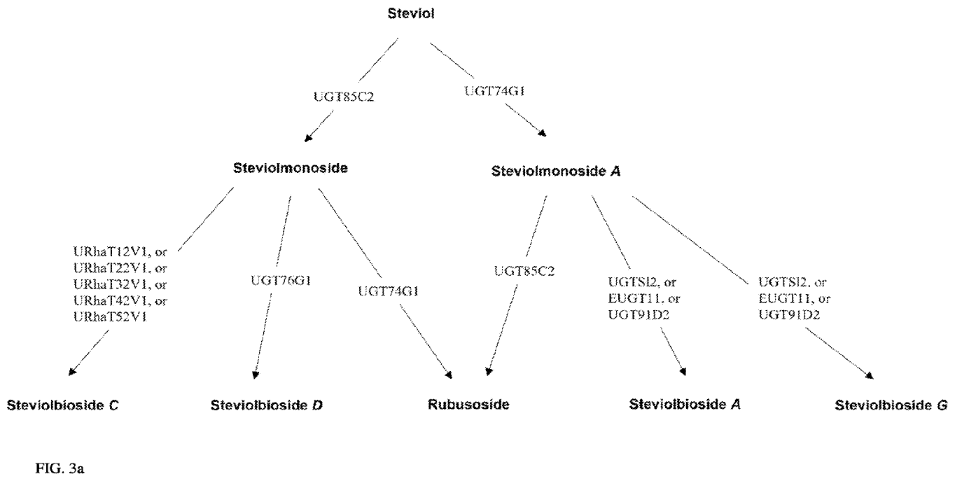

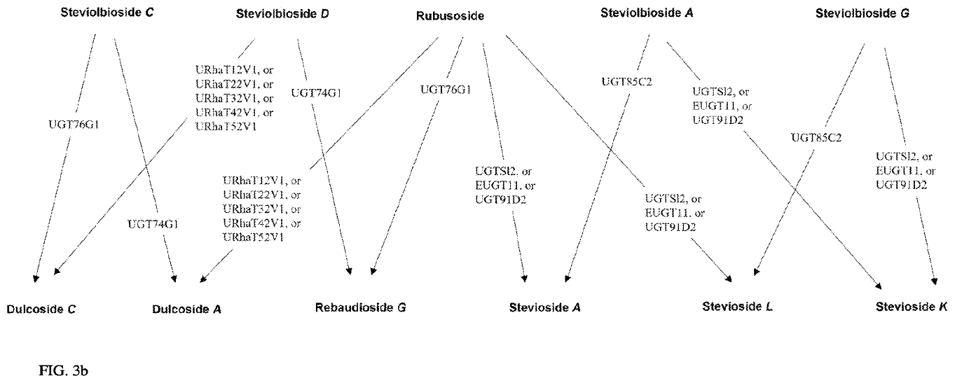

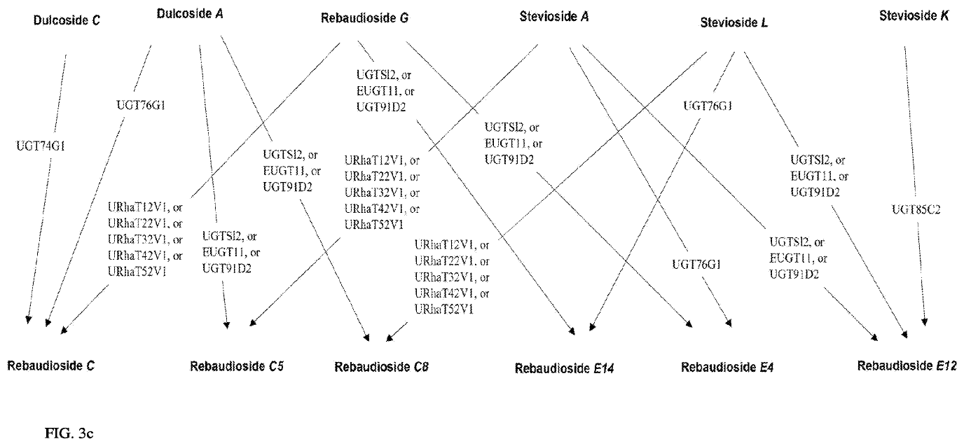

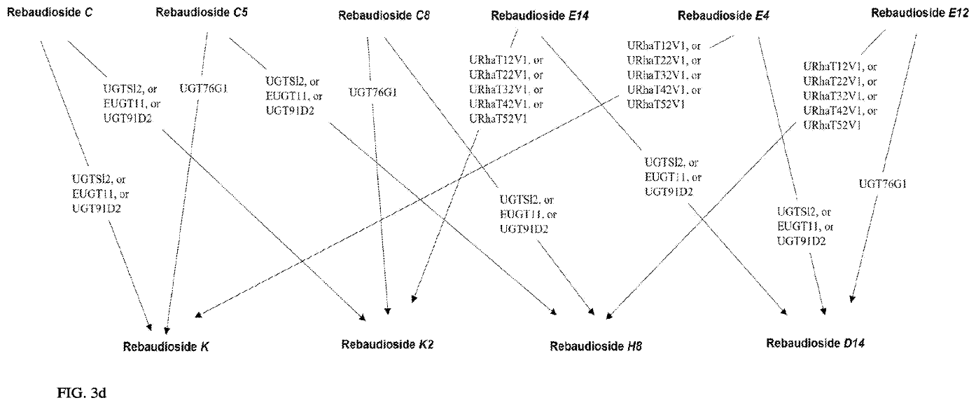

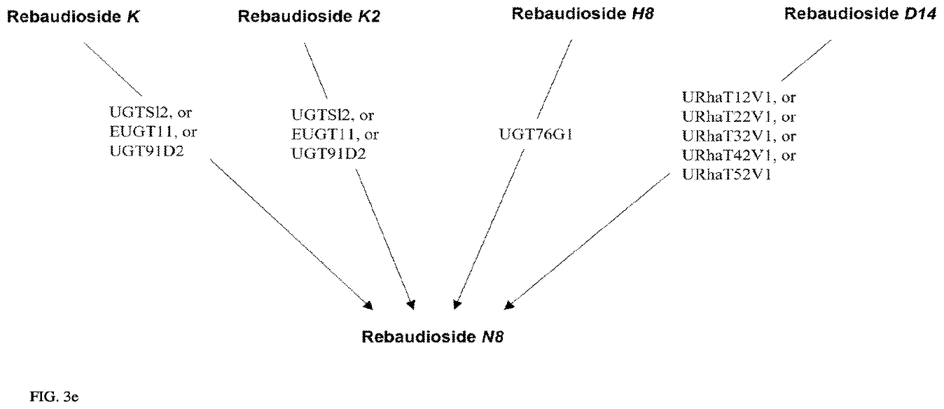

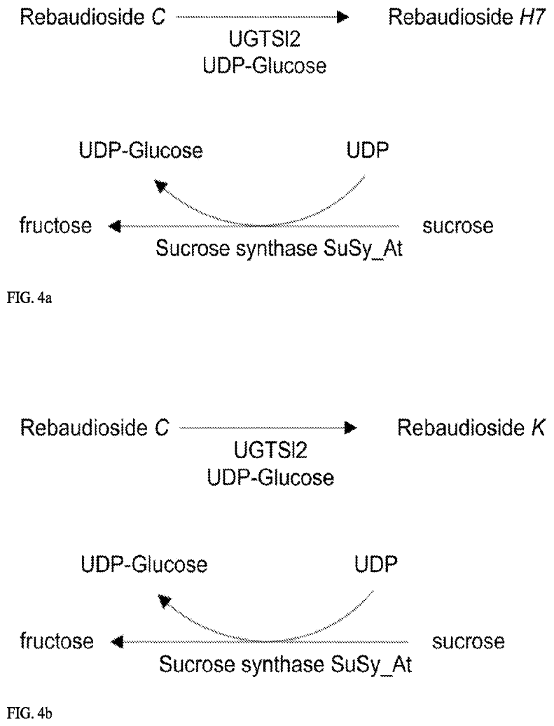

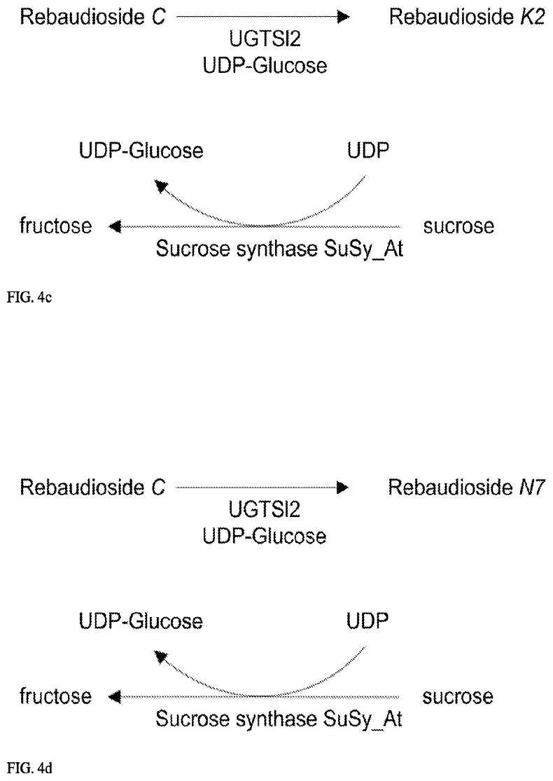

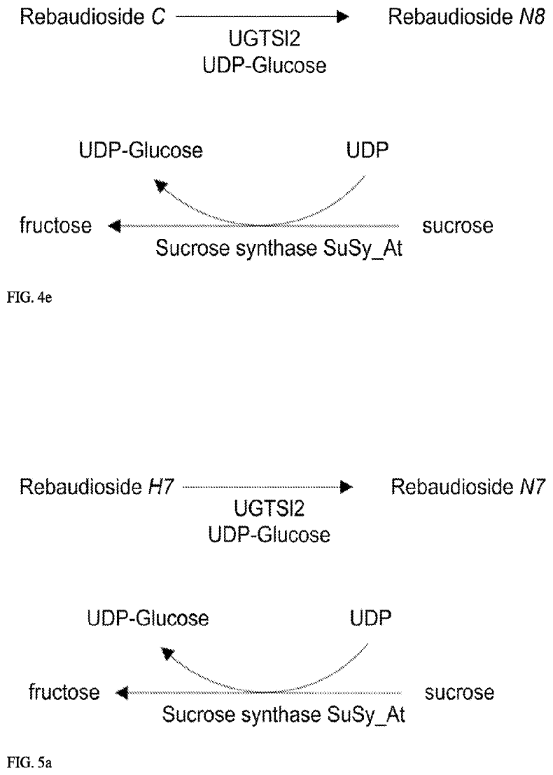

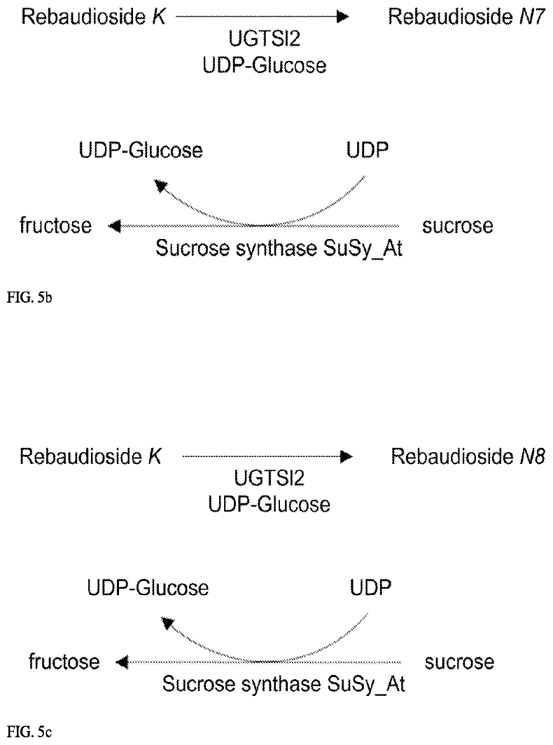

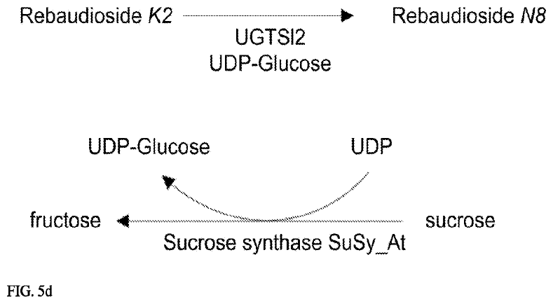

### DETAILED DESCRIPTION

The present invention provides a process for preparing a composition comprising a target steviol glycoside by contacting a starting composition comprising an organic substrate with a microbial cell and/or enzyme preparation, thereby producing a composition comprising a target steviol glycoside.

One object of the invention is to provide an efficient biocatalytic method for preparing target steviol glycosides, particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7, rebaudioside N8, and/or other synthetic steviol glycoside from various starting compositions.

### Starting Composition

As used herein, “starting composition” refers to any composition (generally an aqueous solution) containing one or more organic compound comprising at least one carbon atom.

In one embodiment, the starting composition is selected from the group consisting of steviol, steviol glycosides, polyols and various carbohydrates.

The starting composition steviol glycoside is selected from the group consisting of steviol, steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, or steviol glycosides occurring in Stevia rebaudiana plant, synthetic steviol glycosides, e.g. enzymatically glucosylated steviol glycosides, and combinations thereof.

In one embodiment, the starting composition is steviol.

In another embodiment, the starting composition steviol glycoside is steviolmonoside.

In yet another embodiment, the starting composition steviol glycoside is steviolmonoside A.

In another embodiment, the starting composition steviol glycoside is steviolbioside A.

In another embodiment, the starting composition steviol glycoside is steviolbioside C.

In another embodiment, the starting composition steviol glycoside is steviolbioside D.

In another embodiment, the starting composition steviol glycoside is steviolbioside G.

In another embodiment, the starting composition steviol glycoside is rubusoside.

In another embodiment, the starting composition steviol glycoside is stevioside A.

In another embodiment, the starting composition steviol glycoside is stevioside K.

In another embodiment, the starting composition steviol glycoside is stevioside L.

In another embodiment, the starting composition steviol glycoside is stevioside M.

In another embodiment, the starting composition steviol glycoside is ducolside A.

In another embodiment, the starting composition steviol glycoside is ducolside C.

In another embodiment, the starting composition steviol glycoside is rebaudioside G.

In another embodiment, the starting composition steviol glycoside is rebaudioside C.

In another embodiment, the starting composition steviol glycoside is rebaudioside C5.

In another embodiment, the starting composition steviol glycoside is rebaudioside C7.

In another embodiment, the starting composition steviol glycoside is rebaudioside C8.

In another embodiment, the starting composition steviol glycoside is rebaudioside E4.

In another embodiment, the starting composition steviol glycoside is rebaudioside E12.

In another embodiment, the starting composition steviol glycoside is rebaudioside E13.

In another embodiment, the starting composition steviol glycoside is rebaudioside E14.

In another embodiment, the starting composition steviol glycoside is rebaudioside D13.

In another embodiment, the starting composition steviol glycoside is rebaudioside D14.

In another embodiment, the starting composition steviol glycoside is rebaudioside H7.

In another embodiment, the starting composition steviol glycoside is rebaudioside H8.

In another embodiment, the starting composition steviol glycoside is rebaudioside K.

In another embodiment, the starting composition steviol glycoside is rebaudioside K2.

The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively.

Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced.

The term “carbohydrate” refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, 5-ketofructose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

The starting composition may be synthetic or purified (partially or entirely), commercially available or prepared.

In one embodiment, the starting composition is glycerol.

In another embodiment, the starting composition is glucose.

In another embodiment, the starting composition is rhamnose.

In still another embodiment, the starting composition is sucrose.

In yet another embodiment, the starting composition is starch.

In another embodiment, the starting composition is maltodextrin.

In yet another embodiment, the starting composition is cellulose.

In still another embodiment, the starting composition is amylose.

The organic compound(s) of starting composition serve as a substrate(s) for the production of the target steviol glycoside(s), as described herein.

### Target Steviol Glycoside

The target steviol glycoside of the present method can be any steviol glycoside that can be prepared by the process disclosed herein. In one embodiment, the target steviol glycoside is selected from the group consisting of steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7, rebaudioside N8, or other synthetic steviol glycosides, e.g. enzymatically glucosylated steviol glycosides and combinations thereof.

In one embodiment, the target steviol glycoside is steviolmonoside.

In another embodiment, the target steviol glycoside is steviolmonoside A.

In another embodiment, the target steviol glycoside is steviolbioside A.

In another embodiment, the target steviol glycoside is steviolbioside C.

In another embodiment, the target steviol glycoside is steviolbioside D.

In another embodiment, the target steviol glycoside is steviolbioside G.

In another embodiment, the target steviol glycoside is rubusoside.

In another embodiment, the target steviol glycoside is stevioside A.

In another embodiment, the target steviol glycoside is stevioside K.

In another embodiment, the target steviol glycoside is stevioside L.

In another embodiment, the target steviol glycoside is stevioside M.

In another embodiment, the target steviol glycoside is dulcoside A.

In another embodiment, the target steviol glycoside is dulcoside C.

In another embodiment, the target steviol glycoside is rebaudioside G.

In another embodiment, the target steviol glycoside is rebaudioside C.

In another embodiment, the target steviol glycoside is rebaudioside C5.

In another embodiment, the target steviol glycoside is rebaudioside C7.

In another embodiment, the target steviol glycoside is rebaudioside C8.

In another embodiment, the target steviol glycoside is rebaudioside E4.

In another embodiment, the target steviol glycoside is rebaudioside E12.

In another embodiment, the target steviol glycoside is rebaudioside E13.

In another embodiment, the target steviol glycoside is rebaudioside E14.

In another embodiment, the target steviol glycoside is rebaudioside D13.

In another embodiment, the target steviol glycoside is rebaudioside D14.

In another embodiment, the target steviol glycoside is rebaudioside H7.

In another embodiment, the target steviol glycoside is rebaudioside H8.

In another embodiment, the target steviol glycoside is rebaudioside K.

In another embodiment, the target steviol glycoside is rebaudioside K2.

In another embodiment, the target steviol glycoside is rebaudioside N7.

In another embodiment, the target steviol glycoside is rebaudioside N8.

The target steviol glycoside can be in any polymorphic or amorphous form, including hydrates, solvates, anhydrous or combinations thereof.

In one embodiment, the present invention is a biocatalytic process for the production of steviolmonoside.

In one embodiment, the present invention is a biocatalytic process for the production of steviolmonoside A.

In one embodiment, the present invention is a biocatalytic process for the production of steviolbioside A.

In one embodiment, the present invention is a biocatalytic process for the production of steviolbioside C.

In one embodiment, the present invention is a biocatalytic process for the production of steviolbioside D.

In one embodiment, the present invention is a biocatalytic process for the production of steviolbioside G.

In one embodiment, the present invention is a biocatalytic process for the production of rubusoside.

In one embodiment, the present invention is a biocatalytic process for the production of stevioside A.

In one embodiment, the present invention is a biocatalytic process for the production of stevioside K.

In one embodiment, the present invention is a biocatalytic process for the production of stevioside L.

In one embodiment, the present invention is a biocatalytic process for the production of stevioside M.

In one embodiment, the present invention is a biocatalytic process for the production of dulcoside A.

In one embodiment, the present invention is a biocatalytic process for the production of dulcoside C.

In one embodiment, the present invention is a biocatalytic process for the production of rebaudioside G.

In one embodiment, the present invention is a biocatalytic process for the production of rebaudioside C.

In one embodiment, the present invention is a biocatalytic process for the production of rebaudioside C5.

In one embodiment, the present invention is a biocatalytic process for the production of rebaudioside C7.

In one embodiment, the present invention is a biocatalytic process for the production of rebaudioside C8.

In one embodiment, the present invention is a biocatalytic process for the production of rebaudioside E4.

In one embodiment, the present invention is a biocatalytic process for the production of rebaudioside E12.

In one embodiment, the present invention is a biocatalytic process for the production of rebaudioside E13.

In one embodiment, the present invention is a biocatalytic process for the production of rebaudioside E14.

In one embodiment, the present invention is a biocatalytic process for the production of rebaudioside D13.

In one embodiment, the present invention is a biocatalytic process for the production of rebaudioside D14.

In one embodiment, the present invention is a biocatalytic process for the production of rebaudioside H7.

In one embodiment, the present invention is a biocatalytic process for the production of rebaudioside H8.

In one embodiment, the present invention is a biocatalytic process for the production of rebaudioside K.

In one embodiment, the present invention is a biocatalytic process for the production of rebaudioside K2.

In one embodiment, the present invention is a biocatalytic process for the production of rebaudioside N7.

In one embodiment, the present invention is a biocatalytic process for the production of rebaudioside N8.

In a particular embodiment, the present invention provides for the biocatalytic process for the production of rebaudioside H7 from a starting composition comprising rebaudioside C and UDP-glucose.

In a particular embodiment, the present invention provides for the biocatalytic process for the production of rebaudioside K from a starting composition comprising rebaudioside C and UDP-glucose.

In a particular embodiment, the present invention provides for the biocatalytic process for the production of rebaudioside K2 from a starting composition comprising rebaudioside C and UDP-glucose.

In a particular embodiment, the present invention provides for the biocatalytic process for the production of rebaudioside N7 from a starting composition comprising rebaudioside C and UDP-glucose.

In a particular embodiment, the present invention provides for the biocatalytic process for the production of rebaudioside N7 from a starting composition comprising rebaudioside H7 and UDP-glucose.

In a particular embodiment, the present invention provides for the biocatalytic process for the production of rebaudioside N7 from a starting composition comprising rebaudioside K and UDP-glucose.

In a particular embodiment, the present invention provides for the biocatalytic process for the production of rebaudioside N8 from a starting composition comprising rebaudioside C and UDP-glucose.

In a particular embodiment, the present invention provides for the biocatalytic process for the production of rebaudioside N8 from a starting composition comprising rebaudioside K and UDP-glucose.

In a particular embodiment, the present invention provides for the biocatalytic process for the production of rebaudioside N8 from a starting composition comprising rebaudioside K2 and UDP-glucose.

Optionally, the method of the present invention further comprises separating the target steviol glycoside from the medium to provide a highly purified target steviol glycoside composition. The target steviol glycoside can be separated by any suitable method, such as, for example, crystallization, separation by membranes, centrifugation, extraction, chromatographic separation or a combination of such methods.

In particular embodiments, the process described herein results in a highly purified target steviol glycoside composition. The term “highly purified”, as used herein, refers to a composition having greater than about 80% by weight of the target steviol glycoside on an anhydrous (dried) basis. In one embodiment, the highly purified target steviol glycoside composition contains greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside H7, the process described herein provides a composition having greater than about 80% rebaudioside H7 content by weight on a dried basis. In another particular embodiment, when the target steviol glycoside is rebaudioside H7, the process described herein provides a composition comprising greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside K, the process described herein provides a composition having greater than about 80% rebaudioside K content by weight on a dried basis. In another particular embodiment, when the target steviol glycoside is rebaudioside K, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside K2, the process described herein provides a composition having greater than about 80% rebaudioside K2 content by weight on a dried basis. In another particular embodiment, when the target steviol glycoside is rebaudioside K2, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside N7, the process described herein provides a composition having greater than about 80% rebaudioside N7 content by weight on a dried basis. In another particular embodiment, when the target steviol glycoside is rebaudioside N7, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside N8, the process described herein provides a composition having greater than about 80% rebaudioside N8 content by weight on a dried basis. In another particular embodiment, when the target steviol glycoside is rebaudioside N8, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

### Microorganisms and Enzyme Preparations

In one embodiment of present invention, a microorganism (microbial cell) and/or enzyme preparation is contacted with a medium containing the starting composition to produce target steviol glycosides.

The enzyme can be provided in the form of a whole cell suspension, a crude lysate, a purified enzyme or a combination thereof. In one embodiment, the biocatalyst is a purified enzyme capable of converting the starting composition to the target steviol glycoside. In another embodiment, the biocatalyst is a crude lysate comprising at least one enzyme capable of converting the starting composition to the target steviol glycoside. In still another embodiment, the biocatalyst is a whole cell suspension comprising at least one enzyme capable of converting the starting composition to the target steviol glycoside.

In another embodiment, the biocatalyst is one or more microbial cells comprising enzyme(s) capable of converting the starting composition to the target steviol glycoside. The enzyme can be located on the surface of the cell, inside the cell or located both on the surface of the cell and inside the cell.

In one embodiment of present invention, target steviol glycosides can be produced through fermentation process. In a particular embodiment, fermentation process can utilize a non-genetically modified or a genetically modified organism that is capable of producing one or more steviol glycosides, such as Reb M and Reb D. In another particular embodiment, a genetically engineered microbial strain contains a set of enzymes that can synthesize one or more of target steviol glycosides. One or more steviol glycosides other than target steviol glycosides can also be produced by the genetically engineered microbial strains or enzyme composition prepared from the genetically engineered microbial strains.

In one embodiment, a genetically engineered microbial strain can be used for production of steviol glycosides by expressing various enzymes such as geranylgeranyl diphosphate synthase (GGPPS), ent-copalyl diphosphate synthase (CDPS), kaurene oxidase (KO), kaurene synthase (KS), steviol synthase (KAH), cytochrome P450 reductase (CPR), UGT74G1, UGT85C, UGT76G1, EUGT11 and UGT91D2. WO2014/122227, the entire content of which is hereby incorporated by reference, describes a genetically engineered yeast strain that expresses these enzymes.

In one embodiment, the genetically engineered microbial strain additionally expresses enzymes including UGTS12, URhaT12V1, URhaT22V1, URhaT32V1, URhaT42V1, URhaT52V1 and sucrose synthase (SuSy). In a particular embodiment, UDP-glucosyltransferases are genes that encode polypeptides capable of carrying out reactions such as (i) glucosylation of the —OH functional group at the C13 of a steviol or steviol glycoside, (ii) glucosylation of the —COOH functional group at the C19 of a steviol or steviol glycoside, (iii) beta 1,2-glucosylation at the C2′ of the 19-O-glucose of a steviol glycoside, (iv) beta 1,2-glucosylation at the C2′ of the 13-O-glucose of a steviol glycoside, (v) beta 1,3-glucosylation at the C3′ of the 19-O-glucose of a steviol glycoside, (vi) beta 1,3-glucosylation at the C3′ of the 13-O-glucose of a steviol glycoside, (vii) beta 1,4-glucosylation at the C4′ of the 19-O-glucose of a steviol glycoside, (viii) beta 1,4-glucosylation at the C4′ of the 13-O-glucose of a steviol glycoside, (ix) beta 1,6-glucosylation at the C6′ of the 19-O-glucose of a steviol glycoside and (x) beta 1,6-glucosylation at the C6′ of the 13-O-glucose of a steviol glycoside. For example, UGT85C2 carries out (i); UGT74G1 carries out (ii); UGTS12 carries out (iii), (iv), (vii), (viii), (ix) and (x); UGT76G1 carries out (v), (vi), (vii) and (viii); EUGT11 carries out (iii), (iv), (vii), (viii), (ix) and (x); UGT91D2 carries out (iii), (iv), (vii), (viii), (ix) and (x). In another particular embodiment, UDP-rhamnosyltransferases are genes that encode polypeptides capable of carrying out reactions such as (a) alpha 1,2-rhamnosylation at the C2′ of the 19-O-glucose of a steviol glycoside and (b) alpha 1,2-rhamnosylation at the C2′ of the 13-O-glucose of a steviol glycoside. For example, URhaT12V1 carries out (a) and (b); URhaT22V1 carries out (a) and (b); URhaT32V1 carries out (a) and (b); URhaT42V1 carries out (a) and (b); URhaT52V1 carries out (a) and (b).

In another embodiment, a genetically engineered yeast can be used for production of steviol glycosides includes the following genes encoding a polypeptide capable of (i) synthesizing geranylgeranyl pyrophosphate (GGPP) from farnesyl diphosphate (FPP) and isopentenyl diphosphate (IPP), e.g. geranylgeranyl diphosphate synthase (GGPPS); (ii) synthesizing ent-copalyl diphosphate from GGPP, e.g. ent-copalyl diphosphate synthase (CDPS); (iii) synthesizing ent-kaurene from ent-copalyl pyrophosphate, e.g. kaurene synthase (KS); (iv) synthesizing ent-kaurenoic acid from ent-kaurene, e.g. kaurene oxidase (KO); (v) synthesizing steviol from ent-kaurenoic acid, e.g. steviol synthase (KAH); and converting NADPH to NADP+, e.g. cytochrome P450 reductase (CPR). WO2014/122227, the content of which is hereby incorporated by reference, describes a genetically engineered yeast strain that expresses these enzymes.

In one embodiment, fermentation process can be conducted using any medium at any condition that is useful for production of target steviol glycosides. In the same mixture produced by the fermentation, other steviol glycosides can be produced by the genetically engineered microbial strain, such as Reb M, Reb D, Reb A, etc. Fermentation process generally can be carried out under conditions with presence of oxygen (aerobic conditions), a carbon source, a nutritious medium (nitrogen base) and at a neutral or lower pH. Fermentation process can be a continuous or fed batch process.

In another embodiment, fermentation process is generally conducted by growing a genetically engineered microbial strain in a base medium followed by a longer feeding phase using feed medium mainly consists of glucose or sucrose with traces of metals, vitamins and salts. The fermentation medium consists of >5 g/L glucose or sucrose, >5 g/L ammonium sulfate, >3 g/L potassium dihydrogenphosphate, >0.5 g/L magnesium sulphate, trace elements and vitamins. Verduyn, C. et al. (1992) Yeast 8, 501-517, the content of which is hereby incorporated by reference, describes a minimal composition of a fermentation medium. The fermentation medium can be maintained at about pH 5 and temperature at 30° C.

In another embodiment, fermentation can be conducted in media containing steviol or steviol glycoside(s). One or more genetically engineered microbial strain(s) to be used with the media expresses genes encoding a functional UGT74G1, a functional UGT85C, a functional UGTS12, a functional UGT76G1, a functional EUGT11, a functional UGT91D2, a functional URhaT12V1, a functional URhaT22V1, a functional URhaT32V1, a functional URhaT42V1 and a functional URhaT52V1. Target steviol glycosides, Reb A, Reb M and Reb D may be synthesized in the fermentation media.

In one embodiment, target steviol glycosides can be produced using an enzyme composition prepared from one or more genetically engineered microbial strain(s). In a particular embodiment, the genetically engineered microbial strain(s) expresses genes encoding geranylgeranyl diphosphate synthase (GGPPS), ent-copalyl diphosphate synthase (CDPS), kaurene oxidase (KO), kaurene synthase (KS), steviol synthase (KAH), cytochrome P450 reductase (CPR), sucrose synthase (SuSy), UGT74G1, UGT85C, UGTS12, UGT76G1, EUGT11, UGT91D2, URhaT12V1, URhaT22V1, URhaT32V1, URhaT42V1 and URhaT52V1 enzymes is used for enzyme composition preparation. In another particular embodiment, the organisms can be treated with reagents that disrupt cell membranes to release the enzymes into a composition. In another particular embodiment, the enzymes are secreted into the fermentation media which can be used to prepare the enzyme composition. Various enzymes in the composition react with one or more precursor compounds, e.g. steviol or steviol glycoside(s) through one or multiple enzymatic reaction(s) involving a series of intermediates, to provide a composition consisting of one or more of target steviol glycosides.

In another embodiment, an enzyme composition can be prepared from one cellular extract or combination of multiple cellular extracts from one genetically engineered strain or multiple genetically engineered strains. Each strain expresses a number of enzymes for the bioconversion of one or more of steviol glycoside precursors to one or more of target steviol glycosides and/or other steviol glycosides.

In one embodiment, after fermentation, a composition consisting of steviol glycosides including one or more of target steviol glycosides can be obtained from the culture media using various methods. A permeabilizing agent can be used to enhance the release of the steviol glycosides from the cell into the fermentation media. The genetically engineered microbial strains can be separated from the fermentation media by centrifugation or filtration. Optionally, membrane dialysis can be carried out to remove low molecular weight components such as glucose, basic nutrients and salts. Depending on the application, the obtained composition containing one or more of target steviol glycosides and/or other steviol glycosides can be used.

In one embodiment, for fermentation process that produces enzyme composition extracellularly into the fermentation media, the enzyme composition can be recovered from the growth media using various methods. A permeabilizing agent can be used to enhance the release of enzymes from the cells into the fermentation media. The genetically engineered microbial strains can be separated from the fermentation media by centrifugation or filtration. Optionally, membrane dialysis can be carried out to remove low molecular weight components such as glucose, basic nutrients and salts. The obtained solution containing enzyme composition can be used for bioconversion of one or more of steviol glycoside precursors to one or more of target steviol glycosides and/or other steviol glycosides.

In one embodiment, for fermentation process with the enzyme composition remained intracellularly in the genetically engineered microbial strains, the cells can be obtained from the fermentation media by centrifugation or filtration. A lysis buffer can be used to disrupt cell membrane for releasing enzymes from the cells. The lysed cells can be separated from the solution containing enzyme composition by centrifugation or filtration. The solution containing enzyme composition can be used for bioconversion of one or more of steviol glycoside precursors to one or more of target steviol glycosides and/or other steviol glycosides.

In one embodiment, bioconversion process generally can be carried out by the enzyme composition prepared to convert one or more of steviol glycoside precursors to one or more steviol glycosides under conditions with presence of substrate, UDP-glucose or UDP-rhamnose, ADP-glucose or ADP-rhamnose, at pH around 6-7 and at a temperature of around 45-55° C. The bioconversion substrate can be glucose, sucrose, maltodextrin or liquefied starch. Bioconversion process can be a continuous or fed batch process. The product of bioconversion is a composition consists of one or more of target steviol glycosides and/or other steviol glycosides which can be used as is or further enriched or purified.

In another embodiment, a composition with steviol glycosides including one or more of target steviol glycosides in enriched or purified form can be prepared by further purification. In yet another embodiment, one or more of target steviol glycosides are separated from other steviol glycosides or separated from one another. Such enrichment or purification of steviol glycosides compounds can be carried out on a composition containing one or more of target steviol glycosides in liquid or dry form. Dry form of a composition containing one or more of target steviol glycosides can be obtained by spray drying, flash drying, oven drying or lyophilization.

In one embodiment, a dried material containing one or more of target steviol glycosides is used as a starting material for purification. The dried material containing one or more of target steviol glycosides can be dissolved in a solvent or combination of solvents. An exemplary combination of solvents is a mixture of water and an alcohol (water:alcohol ratio from 1:99 to 99:1) for dissolving the dried material. The dissolution of the dried material in the solvent can be facilitated by heating the mixture at a temperature above room temperature, such as 40-80° C. and mechanical disruption by sonication. The undissolved material can be filtered using a micron or sub-micron filter to produce a solution containing one or more of target steviol glycosides which can be further purified by chromatographic system.

In one embodiment, a dried material containing one or more of target steviol glycosides is used as a starting material for purification. The dried material containing one or more of target steviol glycosides can be suspended in a solvent or combination of solvents. An exemplary combination of solvents is a mixture of water and an alcohol (water:alcohol ratio from 1:99 to 99:1) for suspending the dried material. The suspension of the dried material in the solvent can be facilitated by heating the mixture at a temperature above room temperature, such as 40-80° C. and mechanical disruption by sonication. The undissolved material can be filtered or centrifuged to produce a cake containing one or more of target steviol glycosides which can be further dried to obtain a dry form of a purified composition containing one or more of target steviol glycosides by spray drying, flash drying, oven drying or lyophilization. Optionally, the cake containing one or more of target steviol glycosides can be dissolved and further purified by a chromatographic system.

In one embodiment, the solution containing one or more of target steviol glycosides can be purified by chromatographic system, such as reverse phase liquid chromatography, or multi-column chromatographic system with macroporous adsorption resin, etc. With chromatographic systems, typically a resin is used to adsorb steviol glycoside compounds and remove hydrophilic compounds from the column(s) by using liquid such as water as the mobile phase. Steviol glycosides including one or more of target steviol glycosides can be eluted from the column(s) using a suitable solvent or combination of solvents such as water combined with ethanol, methanol or acetonitrile. The elution of steviol glycosides including one or more of target steviol glycosides from the column(s) can produce a composition which can be used for variety of uses.

In one embodiment, the enzymes necessary for converting the starting composition to target steviol glycosides include the NDP-glucosyltransferases (NGTs), ADP-glucosyltransferases (AGTs), CDP-glucosyltransferases (CGTs), GDP-glucosyltransferases (GGTs), TDP-glucosyltransferases (TGTs), UDP-glucosyltransferases (UGTs). Optionally it may include NDP-recycling enzyme, ADP-recycling enzyme, CDP-recycling enzyme, GDP-recycling enzyme, TDP-recycling enzyme, and/or UDP-recycling enzyme.

In yet another embodiment, the enzymes necessary for converting the starting composition to target steviol glycosides include the NDP-rhamnosyltransferases (NRhaTs), ADP-rhamnosyltransferases (ARhaTs), CDP-rhamnosyltransferases (CRhaTs), GDP-rhamnosyltransferases (GRhaTs), TDP-rhamnosyltransferases (TRhaTs), UDP-rhamnosyltransferases (URhaTs). Optionally it may include NDP-recycling enzyme, ADP-recycling enzyme, CDP-recycling enzyme, GDP-recycling enzyme, TDP-recycling enzyme, and/or UDP-recycling enzyme.

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

The UDP-rhamnosyltransferase can be any UDP-rhamnosyltransferase capable of adding at least one rhamnose unit to steviol and/or a steviol glycoside substrate to provide the target steviol glycoside.

In one embodiment, steviol biosynthesis enzymes, UDP-glucosyltransferases and UDP-rhamnosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases and UDP-rhamnosyltransferases are synthesized.

In one embodiment, the UDP-glucosyltransferase is selected from group including UGT74G1, UGT85C2, UGTS12, UGT76G1 and UGTs having substantial (>85%, >86%, >87%, >88%, >89%, >90%, >91%, >92%, >93%, >94%, >95%, >96%, >97%, >98%, >99%) amino-acid sequence identity to SEQ ID 2, SEQ ID 3, SEQ ID 4 and SEQ ID 5, respectively as well as isolated nucleic acid molecules that code for these UGTs. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to these polypeptides.

In one embodiment, the UDP-rhamnosyltransferase is selected from group including URhaT12V1, URhaT22V1, URhaT32V1, URhaT42V1, URhaT52V1 and URhaTs having substantial (>85%, >86%, >87%, >88%, >89%, >90%, >91%, >92%, >93%, >94%, >95%, >96%, >97%, >98%, >99%) amino-acid sequence identity to SEQ ID 6, SEQ ID 7, SEQ ID 8, SEQ ID 9 and SEQ ID 10, respectively as well as isolated nucleic acid molecules that code for these URhaTs. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to these polypeptides.

In one embodiment, steviol biosynthesis enzymes, UGTs, URhaTs, UDP-glucose recycling system, UDP-rhamnose recycling system and UDP-rhamnose synthesis system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with SEQ ID 2.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In another embodiment, the UDP-rhamnosyltransferase is any UDP-rhamnosyltransferase capable of adding at least one rhamnose unit to the existing glucose at C13 of any starting steviol glycoside to give a target steviol glycoside with at least one additional rhamnose bearing at least one alpha 1→2 rhamnopyranoside glycosidic linkage(s) at the newly formed glycosidic bond(s). In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with SEQ ID 2.

In another embodiment, the UDP-rhamnosyltransferase is any UDP-rhamnosyltransferase capable of adding at least one rhamnose unit to steviolmonoside to form steviolbioside C. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to steviolmonoside to form steviolbioside D. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolmonoside A to form steviolbioside G. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolbioside C to form dulcoside C. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolbioside C to form dulcoside A. In a particular embodiment, the UDP-glucosyltransferase is UGT74G1 or a UGT having >85% amino-acid sequence identity with SEQ ID 2.

In another embodiment, the UDP-rhamnosyltransferase is any UDP-rhamnosyltransferase capable of adding at least one rhamnose unit to steviolbioside D to form dulcoside C. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to steviolbioside D to form rebaudioside G. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolbioside D to form stevioside M. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rubusoside to form dulcoside A. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rubusoside to form rebaudioside G. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside A. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rubusoside to form stevioside L. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside A to form stevioside K. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to steviolbioside G to form stevioside L. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside G to form stevioside K. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to dulcoside C to form rebaudioside C. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to dulcoside C to form rebaudioside C7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to dulcoside A to form rebaudioside C. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to dulcoside A to form rebaudioside C5. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to dulcoside A to form rebaudioside C8. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside G to form rebaudioside C. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside G to form rebaudioside E13. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside G to form rebaudioside E14. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside G to form rebaudioside E4. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to stevioside M to form rebaudioside C7. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to stevioside M to form rebaudioside E13. In a particular embodiment, the UDP-glucosyltransferase is UGT74G1 or a UGT having >85% amino-acid sequence identity with SEQ ID 2.

In another embodiment, the UDP-rhamnosyltransferase is any UDP-rhamnosyltransferase capable of adding at least one rhamnose unit to stevioside A to form rebaudioside C5. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to stevioside A to form rebaudioside E4. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E12. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to stevioside L to form rebaudioside C8. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to stevioside L to form rebaudioside E14. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside L to form rebaudioside E12. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to stevioside K to form rebaudioside E12. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to rebaudioside C to form rebaudioside H7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C to form rebaudioside K. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C to form rebaudioside K2. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C7 to form rebaudioside H7. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to rebaudioside C5 to form rebaudioside K. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside C5 to form rebaudioside H8. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C8 to form rebaudioside K2. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside C8 to form rebaudioside H8. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside E13 to form rebaudioside H7. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10. In another embodiment, the UDP-glucosyltransferase is any UDP-glucosyltransferase capable of adding at least one glucose unit to rebaudioside E13 to form rebaudioside D13. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside E4 to form rebaudioside K. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside E4 to form rebaudioside D13. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside E4 to form rebaudioside D14. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside E12 to form rebaudioside H8. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside E12 to form rebaudioside D14. In a particular embodiment, the UDP-glucosyltransferase is UGT76G1, or a UGT having >85% amino-acid sequence identity with SEQ ID 5.

In another embodiment, the UDP-rhamnosyltransferase is any UDP-rhamnosyltransferase capable of adding at least one rhamnose unit to rebaudioside E14 to form rebaudioside K2. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside E14 to form rebaudioside D14. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside H7 to form rebaudioside N7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside H8 to form rebaudioside N8. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside K to form rebaudioside N7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside K to form rebaudioside N8. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside K2 to form rebaudioside N8. In a particular embodiment, the UDP-glucosyltransferase is UGTS12 or a UGT having >85% amino-acid sequence identity with SEQ ID 4.

In another embodiment, the UDP-rhamnosyltransferase is any UDP-rhamnosyltransferase capable of adding at least one rhamnose unit to rebaudioside D13 to form rebaudioside N7. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-rhamnosyltransferase is any UDP-rhamnosyltransferase capable of adding at least one rhamnose unit to rebaudioside D14 to form rebaudioside N8. In another particular embodiment, the UDP-rhamnosyltransferase is URhaT12V1, or a URhaT having >85% amino-acid sequence identity with SEQ ID 6. In yet another particular embodiment, the UDP-rhamnosyltransferase is URhaT22V1, or a URhaT having >85% amino-acid sequence identity with SEQ ID 7. In another particular embodiment, the UDP-rhamnosyltransferase is URhaT32V1, or a URhaT having >85% amino-acid sequence identity with SEQ ID 8. In another particular embodiment, the UDP-rhamnosyltransferase is URhaT42V1, or a URhaT having >85% amino-acid sequence identity with SEQ ID 9. In another particular embodiment, the UDP-rhamnosyltransferase is URhaT52V1, or a URhaT having >85% amino-acid sequence identity with SEQ ID 10.

In another embodiment, the UDP-glucosyltransferase is any UDP-glucosyltransferase capable of adding at least one glucose unit to rebaudioside C to form rebaudioside N7. In a particular embodiment, the UDP-glucosyltransferase is UGTS12 or a UGT having >85% amino-acid sequence identity with SEQ ID 4.

In another embodiment, the UDP-glucosyltransferase is any UDP-glucosyltransferase capable of adding at least one glucose unit to rebaudioside C to form rebaudioside N8. In a particular embodiment, the UDP-glucosyltransferase is UGTS12 or a UGT having >85% amino-acid sequence identity with SEQ ID 4.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose. The UDP recycling enzyme can be sucrose synthase SuSy_At or a sucrose synthase having >85% amino-acid sequence identity with SuSy_At and the recycling substrate can be sucrose.

Optionally, the method of the present invention further comprises recycling UDP to provide UDP-rhamnose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-rhamnosyltransferase and UDP-rhamnose. The recycling substrate can be rhamnosyl-containing residue.

In one embodiment, UDP-rhamnose can be synthesized from UDP-glucose by UDP-rhamnose synthase system. In another embodiment, UDP-rhamnose can be synthesized from UDP-glucose by trifunctional UDP-rhamnose synthase NRF1 or NR32, as it is described in WO2020/205685A1, the content of which is hereby incorporated by reference, or a UDP-rhamnose synthase having >85% amino-acid sequence identity with NRF1 or NR32.

In one embodiment, the UDP-glucose recycling catalyst is sucrose synthase SuSy_At or a sucrose synthase having >85% amino-acid sequence identity with SuSy_At.

In one embodiment, the recycling substrate for UDP-glucose recycling catalyst is sucrose.

In one embodiment, the UDP-rhamnose synthesis and recycling catalyst is trifunctional UDP-rhamnose synthase NRF1 or NR32 or a UDP-rhamnose synthase having >85% amino-acid sequence identity with NRF1 or NR32. Exemplary synthesis and recycling pathway and gene expression are provided in WO2020/205685A1 and WO2018/190378A1 respectively, the content of which is hereby incorporated by reference.

In one embodiment, the recycling substrate for UDP-rhamnose recycling catalyst is a molecule comprising rhamnosyl residue. In another embodiment, the synthesis substrate for UDP-rhamnose synthesis catalyst is UDP-glucose. Exemplary synthesis and recycling pathway and gene expression are provided in WO2020/205685A1 and WO2018/190378A1 respectively, the content of which is hereby incorporated by reference.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside H7. In another embodiment, the recipient steviol glycoside is rebaudioside K. In another embodiment, the recipient steviol glycoside is rebaudioside K2. In another embodiment, the recipient steviol glycoside is rebaudioside N7.

In another embodiment, the recipient steviol glycoside is rebaudioside N8.

One embodiment of the present invention is a microbial cell comprising an enzyme, i.e. an enzyme capable of converting the starting composition to the target steviol glycoside.

Accordingly, some embodiments of the present method include contacting a microorganism with a medium containing the starting composition to provide a medium comprising at least one target steviol glycoside.

The microorganism can be any microorganism possessing the necessary enzyme(s) for converting the starting composition to target steviol glycoside(s). These enzymes are encoded within the microorganism's genome.

Suitable microorganisms include, but are not limited to, E. coli, Saccharomyces sp., Aspergillus sp., Pichia sp., Bacillus sp., Yarrowia sp. etc.

In one embodiment, the microorganism is free when contacted with the starting composition.

In another embodiment, the microorganism is immobilized when contacted with the starting composition. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In still another embodiment, the enzyme capable of converting the starting composition to the target steviol glycoside is secreted out of the microorganism and into the reaction medium.

The target steviol glycoside is optionally purified. Purification of the target steviol glycoside from the reaction medium can be achieved by at least one suitable method to provide a highly purified target steviol glycoside composition. Suitable methods include crystallization, separation by membranes, centrifugation, extraction (liquid or solid phase), chromatographic separation, HPLC (preparative or analytical) or a combination of such methods.

Uses Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention can be used “as-is” or in combination with other sweeteners, flavors, flavor stabilizers, flavorings with modifying properties (FMP), foaming suppressors, solubility enhancing agents, food ingredients, salts thereof and combinations thereof.

Non-limiting examples of sweeteners include, but are not limited to, steviol glycosides, carbohydrates, psicose, 5-ketofructose, tagatose, polyols, sugar alcohols, natural high intensity sweeteners, synthetic high intensity sweeteners, reduced calorie sweeteners, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin, monatin salts, other indole derivative sweeteners, selligueainA, hematoxylin, monellin, osladin, pterocaryosideA, pterocaryoside B, mabinlin, pentadin, miraculin, curculin, neoculin, chlorogenic acid, cynarin, Luo Han Guo sweetener, mogroside V, siamenoside, siratose, salts thereof, and combinations thereof.

Non-limiting examples of flavors include, but are not limited to, lime, lemon, orange, fruit, banana, grape, pear, pineapple, mango, berry, bitter almond, cola, cinnamon, sugar, cotton candy, vanilla, other compounds listed in FEMA (Flavor Extract Manufacturers Association) flavoring substances GRAS lists, salts thereof, and combinations thereof.

Non-limiting examples of other food ingredients include, but are not limited to, acidulants, organic and amino acids, coloring agents, bulking agents, modified starches, gums, texturizers, preservatives, caffeine, color stabilizers, flavor stabilizers, natural sweetener suppressors, additives, antioxidants, emulsifiers, stabilizers, thickeners, gelling agents, physiologically active substances, functional ingredients, salts thereof, and combinations thereof.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention can be prepared in various polymorphic forms, including but not limited to hydrates, solvates, anhydrous, amorphous forms and combinations thereof.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be incorporated as a flavor stabilizer in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be incorporated as a flavoring with modifying properties (FMP) in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be incorporated as a foam stabilizer in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be incorporated as a solubility enhancing agent in foodstuffs, beverages, pharmaceutical compositions, cosmetics, chewing gums, table top products, cereals, dairy products, toothpastes and other oral cavity compositions, etc.

In some embodiments, the highly purified target glycoside(s) particularly, steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 of present invention are present in consumable products, foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, lipsticks, etc at a concentration from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In one embodiment, the sweetener is present in the beverage in an amount from about 0.0001% by weight to about 8% by weight, such as for example, from about 0.0001% by weight to about 0.0005% by weight, from about 0.0005% by weight to about 0.001% by weight, from about 0.001% by weight to about 0.005% by weight, from about 0.005% by weight to about 0.01% by weight, from about 0.01% by weight to about 0.05% by weight, from about 0.05% by weight to about 0.1% by weight, from about 0.1% by weight to about 0.5% by weight, from about 0.5% by weight to about 1% by weight, from about 1% by weight to about 2% by weight, from about 2% by weight to about 3% by weight, from about 3% by weight to about 4% by weight, from about 4% by weight to about 5% by weight, from about 5% by weight to about 6% by weight, from about 6% by weight to about 7% by weight, and from about 7% by weight to about 8% by weight.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 and/or combinations thereof, obtained according to this invention, may be employed as a sweetening compound as the sole sweetener, or it may be used together with at least one high intensity sweeteners such as dulcoside A, dulcoside B, dulcoside C, dulcoside D, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 11, rebaudioside 1m, rebaudioside 1n, rebaudioside 10, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f, rebaudioside 2g, rebaudioside 2h, rebaudioside 2i, rebaudioside 2j, rebaudioside 2k, rebaudioside 21, rebaudioside 2m, rebaudioside 2n, rebaudioside 2o, rebaudioside 2p, rebaudioside 2q, rebaudioside 2r, rebaudioside 2s, rebaudioside A, rebaudioside A1G, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside C8, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside D14, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside E14, rebaudioside F, rebaudioside F1, rebaudioside F2, rebaudioside F3, rebaudioside F4, rebaudioside F5, rebaudioside G, rebaudioside H, rebaudioside H1, rebaudioside H2, rebaudioside H3, rebaudioside H4, rebaudioside H5, rebaudioside H6, rebaudioside H7, rebaudioside H8, rebaudioside I, rebaudioside 12, rebaudioside 13, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside N8, rebaudioside 0, rebaudioside 02, rebaudioside 03, rebaudioside 04, rebaudioside 05, rebaudioside 06, rebaudioside 07, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside U4, rebaudioside U5, rebaudioside U6, rebaudioside V, rebaudioside V2, rebaudioside V3, rebaudioside VIII, rebaudioside VIIIa, rebaudioside VIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolbioside H, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, stevioside N, SvG7, NSF-02, carbohydrates, psicose, 5-ketofructose, tagatose, allose, erythritol, polyols, sugar alcohols, natural high intensity sweeteners, synthetic high intensity sweeteners, reduced calorie sweeteners, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin, monatin salts, other indole derivative sweeteners, selligueainA, hematoxylin, monellin, osladin, pterocaryosideA, pterocaryoside B, mabinlin, pentadin, miraculin, curculin, neoculin, chlorogenic acid, cynarin, Luo Han Guo sweetener, mogroside V, siamenoside, siratose, salts thereof, and combinations thereof.

In one embodiment, steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used in a sweetener composition comprising a compound selected from the group consisting of dulcoside A, dulcoside B, dulcoside C, dulcoside D, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f, rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s, rebaudioside A, rebaudioside A1G, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside C8, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside D14, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside E14, rebaudioside F, rebaudioside F1, rebaudioside F2, rebaudioside F3, rebaudioside F4, rebaudioside F5, rebaudioside G, rebaudioside H, rebaudioside H1, rebaudioside H2, rebaudioside H3, rebaudioside H4, rebaudioside H5, rebaudioside H6, rebaudioside H7, rebaudioside H8, rebaudioside I, rebaudioside 12, rebaudioside 13, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside N8, rebaudioside 0, rebaudioside 02, rebaudioside 03, rebaudioside 04, rebaudioside 05, rebaudioside 06, rebaudioside 07, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside U4, rebaudioside U5, rebaudioside U6, rebaudioside V, rebaudioside V2, rebaudioside V3, rebaudioside VIII, rebaudioside VIIIa, rebaudioside VIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolbioside H, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, stevioside N, SvG7, NSF-02, carbohydrates, psicose, 5-ketofructose, tagatose, allose, erythritol, polyols, sugar alcohols, natural high intensity sweeteners, synthetic high intensity sweeteners, reduced calorie sweeteners, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin, monatin salts, other indole derivative sweeteners, selligueainA, hematoxylin, monellin, osladin, pterocaryosideA, pterocaryoside B, mabinlin, pentadin, miraculin, curculin, neoculin, chlorogenic acid, cynarin, Luo Han Guo sweetener, mogroside V, siamenoside, siratose, salts thereof, and combinations thereof.

Highly purified target glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and combinations thereof.

Moreover, highly purified target steviol glycoside(s) particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used in combination with natural sweetener suppressors such as gymnemic acid, hodulcin, ziziphin, lactisole, and others.

Steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 may also be combined with various umami taste enhancers. Steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be mixed with umami tasting and sweet amino acids such as aspartic acid, glycine, alanine, threonine, proline, serine, glutamate, lysine, tryptophan, salts thereof and combinations thereof.

Highly purified target steviol glycoside(s) particularly, steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers, salts thereof and combinations thereof.

Highly purified target steviol glycoside(s) particularly, steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 may be combined with reduced calorie sweeteners such as, for example, D-psicose, 5-ketofructose, D-tagatose, L-sugars, L-sorbose, L-arabinose and combinations thereof.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, 5-ketofructose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols, polyols, prebiotics, probiotics, postbiotics, phytoestrogens, soy protein, sulfides/thiols, amino acids, proteins, vitamins, and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used as a sweetener not only for drinks, foodstuffs, and other products dedicated for human consumption, but also in animal feed and fodder with improved characteristics.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be applied as a flavor stabilizer to produce zero calorie, reduced calorie or diabetic beverages and food products with improved flavor stability compared to a control product that does not contain the glycoside(s). It may also be used in drinks, foodstuffs, pharmaceuticals, and other products in which flavor stabilization is preferred. In addition, highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used as a flavor stabilizer not only for drinks, foodstuffs, and other products dedicated for human consumption, but also in animal feed and fodder with improved characteristics.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be applied as a flavoring with modifying properties (FMP) to produce zero calorie, reduced calorie or diabetic beverages and food products with modification (including enhancing or suppressing) of flavor and/or taste profile. It may also be used in drinks, foodstuffs, pharmaceuticals, and other products in which modification (including enhancing or suppressing) of flavor and/or taste profile is preferred. In addition, highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used as a flavoring with modifying properties (FMP) not only for drinks, foodstuffs, and other products dedicated for human consumption, but also in animal feed and fodder with improved characteristics.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be applied as a foaming suppressor to produce zero calorie, reduced calorie or diabetic beverages and food products with suppressed foaming. It may also be used in drinks, foodstuffs, pharmaceuticals, and other products in which foaming suppression is preferred. In addition, highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used as a foaming suppressor not only for drinks, foodstuffs, and other products dedicated for human consumption, but also in animal feed and fodder with improved characteristics.

Highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be applied as a solubility enhancing agent to produce zero calorie, reduced calorie or diabetic beverages and food products having less insoluble material compared to a control product that does not contain the glycoside(s). It may also be used in drinks, foodstuffs, pharmaceuticals, and other products in which solubility enhancement of insoluble material is preferred. In addition, highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 can be used as a solubility enhancing agent not only for drinks, foodstuffs, and other products dedicated for human consumption, but also in animal feed and fodder with improved characteristics.

Examples of consumable products in which highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained according to this invention may be used as a sweetening compound, flavor stabilizer, flavoring with modifying properties (FMP), foaming suppressor and/or solubility enhancing agent include, but are not limited to, consumable products, foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, lipsticks, and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

Moreover, the highly purified target steviol glycoside(s) steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 obtained in this invention may be used in dry or liquid forms.

The highly purified target steviol glycoside can be added before or after heat treatment of food products. The amount of the highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 depends on the purpose of usage. As discussed above, it can be added alone or in combination with other compounds.

The present invention is also directed to sweetness enhancement in beverages using steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 as a sweetness enhancer, wherein steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 is present in a concentration at or below their respective sweetness recognition thresholds.

The present invention is also directed to flavor stabilization of food and beverages using steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 as a flavor stabilizer, wherein steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 is present in a concentration that allows for flavor stabilization of products.

The present invention is also directed to modification (including enhancing or suppressing) of flavor and/or taste profile of food and beverages using steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 as a flavoring with modifying properties (FMP), wherein steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 is present in a concentration that allows for modification (including enhancing or suppressing) of flavor and/or taste profile of products.

The present invention is also directed to foaming suppression of food and beverages using steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 as a foaming suppressor, wherein steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 is present in a concentration that allows for foaming suppression of products.

The present invention is also directed to solubility enhancement of insoluble material in food and beverages using steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 as a solubility enhancing agent, wherein steviolmonoside, steviolmonosideA, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 is present in a concentration that allows for solubility enhancement of insoluble material in products.

As used herein, the term “sweetness enhancer” refers to a compound or a mixture of compounds capable of enhancing or intensifying the perception of sweet taste in food and beverage products. The term “sweetness enhancer” is synonymous with the terms “sweet taste potentiator,” “sweetness potentiator,” “sweetness amplifier,” and “sweetness intensifier.”

As used herein, the term “flavor stabilizer” refers to a compound or a mixture of compounds capable of stabilizing the flavor in food and beverage products. It is contemplated that a flavor stabilizer can be used alone, or in combination with other flavor stabilizers.

As used herein, the term “flavoring with modifying properties (FMP)” refers to a compound or a mixture of compounds that enhance, subdue or otherwise affect the taste and/or flavor profile without themselves being sweeteners or flavorings. The Flavor and Extracts Manufacturing Association (FEMA) has developed a protocol published in the November 2013 Edition of Food Technology. It is contemplated that a flavoring with modifying properties (FMP) can be used alone, or in combination with other flavorings.

The term “sweetness recognition threshold concentration” as generally used herein, is the lowest known concentration of a sweet compound that is perceivable by the human sense of taste, typically around 1.0% sucrose equivalence (1.0% SE). Generally, the sweetness enhancers may enhance or potentiate the sweet taste of sweeteners without providing any noticeable sweet taste by themselves when present at or below the sweetness recognition threshold concentration of a given sweetness enhancer; however, the sweetness enhancers may themselves provide sweet taste at concentrations above their sweetness recognition threshold concentration. The sweetness recognition threshold concentration is specific for a particular enhancer and can vary based on the beverage matrix. The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

In some embodiments, sweetener is present in the beverage in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.0001% by weight to about 10% by weight, such as for example, from about 0.0001% by weight to about 0.0005% by weight, from about 0.0005% by weight to about 0.001% by weight, from about 0.001% by weight to about 0.005% by weight, from about 0.005% by weight to about 0.01% by weight, from about 0.01% by weight to about 0.05% by weight, from about 0.05% by weight to about 0.1% by weight, from about 0.1% by weight to about 0.5% by weight, from about 0.5% by weight to about 1% by weight, from about 1% by weight to about 2% by weight, from about 2% by weight to about 3% by weight, from about 3% by weight to about 4% by weight, from about 4% by weight to about 5% by weight, from about 5% by weight to about 6% by weight, from about 6% by weight to about 7% by weight, from about 7% by weight to about 8% by weight, from about 8% by weight to about 9% by weight, or from about 9% by weight to about 10% by weight. In a particular embodiment, the sweetener is present in the beverage in an amount from about 0.5% by weight to about 10% by weight. In another particular embodiment, the sweetener is present in the beverage in an amount from about 2% by weight to about 8% by weight.

In one embodiment, the sweetener is a traditional caloric sweetener. Suitable sweeteners include, but are not limited to, sucrose, fructose, glucose, high fructose corn syrup and high fructose starch syrup.

In another embodiment, the sweetener is erythritol.

In still another embodiment, the sweetener is a rare sugar. Suitable rare sugars include, but are not limited to, D-allose, D-psicose, D-ribose, D-tagatose, L-glucose, L-fucose, L-arabinose, D-turanose, D-leucrose, 5-ketofructose and combinations thereof.

It is contemplated that a sweetener can be used alone, or in combination with other sweeteners.

In one embodiment, the rare sugar is D-allose. In a more particular embodiment, D-allose is present in the beverage in an amount of about 0.5% to about 10% by weight, such as, for example, from about 2% to about 8%.

In another embodiment, the rare sugar is D-psicose. In a more particular embodiment, D-psicose is present in the beverage in an amount of about 0.5% to about 10% by weight, such as, for example, from about 2% to about 8%.

In still another embodiment, the rare sugar is D-ribose. In a more particular embodiment, D-ribose is present in the beverage in an amount of about 0.5% to about 10% by weight, such as, for example, from about 2% to about 8%.

In yet another embodiment, the rare sugar is D-tagatose. In a more particular embodiment, D-tagatose is present in the beverage in an amount of about 0.5% to about 10% by weight, such as, for example, from about 2% to about 8%.

In a further embodiment, the rare sugar is L-glucose. In a more particular embodiment, L-glucose is present in the beverage in an amount of about 0.5% to about 10% by weight, such as, for example, from about 2% to about 8%.

In one embodiment, the rare sugar is L-fucose. In a more particular embodiment, L-fucose is present in the beverage in an amount of about 0.5% to about 10% by weight, such as, for example, from about 2% to about 8%.

In another embodiment, the rare sugar is L-arabinose. In a more particular embodiment, L-arabinose is present in the beverage in an amount of about 0.5% to about 10% by weight, such as, for example, from about 2% to about 8%.

In still another embodiment, the rare sugar is D-turanose. In a more particular embodiment, D-turanose is present in the beverage in an amount of about 0.5% to about 10% by weight, such as, for example, from about 2% to about 8%.

In yet another embodiment, the rare sugar is D-leucrose. In a more particular embodiment, D-leucrose is present in the beverage in an amount of about 0.5% to about 10% by weight, such as, for example, from about 2% to about 8%.

In a further embodiment, the rare sugar is 5-ketofructose. In a more particular embodiment, 5-ketofructose is present in the beverage in an amount of about 0.5% to about 10% by weight, such as, for example, from about 2% to about 8%.

The addition of the sweetness enhancer at a concentration at or below its sweetness recognition threshold increases the detected sucrose equivalence of the beverage comprising the sweetener and the sweetness enhancer compared to a corresponding beverage in the absence of the sweetness enhancer. Moreover, sweetness can be increased by an amount more than the detectable sweetness of a solution containing the same concentration of the at least one sweetness enhancer in the absence of any sweetener.

Accordingly, the present invention also provides a method for enhancing the sweetness of a food or beverage comprising a sweetener comprising providing a food or beverage comprising a sweetener and adding a sweetness enhancer selected from steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 or a combination thereof, wherein steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 are present in a concentration at or below their sweetness recognition thresholds.

Accordingly, the present invention also provides a method for stabilizing the flavor of a food or beverage comprising providing a food or beverage and adding a flavor stabilizer selected from steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 or a combination thereof, wherein steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 are present in a concentration that allows for improved flavor stability compared to a control product that does not contain the flavor stabilizer.

Accordingly, the present invention also provides a method for modification (including enhancing or suppressing) of flavor and/or taste profile of a food or beverage comprising providing a food or beverage and adding a flavoring with modifying properties (FMP) selected from steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 or a combination thereof, wherein steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 are present in a concentration that allows for modification (including enhancing or suppressing) of flavor and/or taste profile.

In one embodiment, the present invention also provides a method for adding steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 in a concentration at or below the sweetness recognition threshold to a food or beverage containing a sweetener to increase the detected sucrose equivalence from about 1.0% to about 5.0%, such as, for example, about 1.0%, about 1.5%, about 2.0%, about 2.5%, about 3.0%, about 3.5%, about 4.0%, about 4.5% or about 5.0%.

In another embodiment, the present invention also provides a method for adding steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8 in a concentration that allows for modification (including enhancing or suppressing) of flavor and/or taste profile to a food or beverage to modify (including enhancing or suppressing) the flavor and/or taste profile.

This invention provides rebaudioside N7 with the following formula:

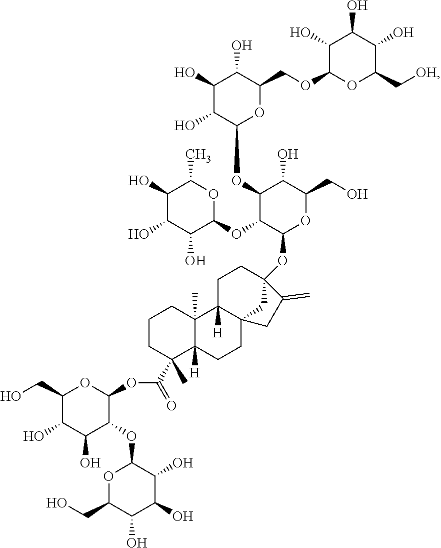

including salts thereof. or combinations thereof.

This invention also provides rebaudioside N8 with the following formula:

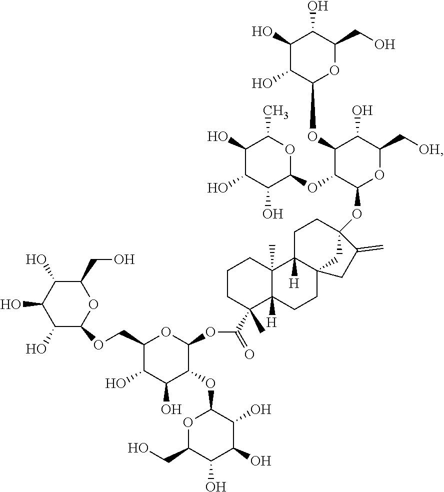

including salts thereof, or combinations thereof.

Furthermore, this disclosure provides a method for producing rebaudioside N7, comprising the steps of providing a starting composition comprising an organic compound with at least one carbon atom; providing an enzyme preparation or microorganism containing at least one enzyme selected from the group consisting of steviol biosynthesis enzymes, NDP-glucosyltransferases and NDP-rhamnosyltransferases and optionally NDP recycling enzymes; contacting the enzyme preparation or microorganism with a medium containing the starting composition to produce a medium comprising rebaudioside N7.

This disclosure also provides a method for producing rebaudioside N8, comprising the steps of providing a starting composition comprising an organic compound with at least one carbon atom; providing an enzyme preparation or microorganism containing at least one enzyme selected from the group consisting of steviol biosynthesis enzymes, NDP-glucosyltransferases and NDP-rhamnosyltransferases and optionally NDP recycling enzymes; contacting the enzyme preparation or microorganism with a medium containing the starting composition to produce a medium comprising rebaudioside N8.

Moreover, this disclosure provides a method for producing rebaudioside N7, comprising the steps of providing a starting composition comprising an organic compound with at least one carbon atom; providing a biocatalyst comprising at least one enzyme selected from the group consisting of steviol biosynthesis enzymes, NDP-glucosyltransferases and NDP-rhamnosyltransferases and optionally NDP recycling enzymes; contacting the biocatalyst with a medium containing the starting composition to produce a medium comprising rebaudioside N7.

This disclosure also provides a method for producing rebaudioside N8, comprising the steps of providing a starting composition comprising an organic compound with at least one carbon atom; providing a biocatalyst comprising at least one enzyme selected from the group consisting of steviol biosynthesis enzymes, NDP-glucosyltransferases and NDP-rhamnosyltransferases and optionally NDP recycling enzymes; contacting the biocatalyst with a medium containing the starting composition to produce a medium comprising rebaudioside N8.

Optionally, the method above further comprises the step of separating rebaudioside N7 from the medium to provide a highly purified composition of rebaudioside N7.

The method above also further comprises the step of separating rebaudioside N8 from the medium to provide a highly purified composition of rebaudioside N8.

In the methods above, the starting composition is selected from the group consisting of steviol, steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, other steviol glycosides, polyols, carbohydrates, and combinations thereof.

In the methods above, the microorganism is selected from the group consisting of E. coli, Saccharomyces sp., Aspergillus sp., Pichia sp., Bacillus sp., and Yarrowia sp.

In the methods above, the biocatalyst is an enzyme, or a cell comprising one or more enzyme, capable of converting the starting composition to rebaudioside N7.

In the methods above, the biocatalyst is an enzyme, or a cell comprising one or more enzyme, capable of converting the starting composition to rebaudioside N8.

The enzyme is selected from the group consisting of a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGTS12, UGT76G1, URhaT12V1, URhaT22V1, URhaT32V1 URhaT42V1 or URhaT52V1 or mutant variant thereof having >85% amino-acid sequence identity, >86% amino-acid sequence identity, >87% amino-acid sequence identity, >88% amino-acid sequence identity, >89% amino-acid sequence identity, >90% amino-acid sequence identity, >91% amino-acid sequence identity, >92% amino-acid sequence identity, >93% amino-acid sequence identity, >94% amino-acid sequence identity, >95% amino-acid sequence identity, >96% amino-acid sequence identity, >97% amino-acid sequence identity, >98% amino-acid sequence identity, >99% amino-acid sequence identity.

The content of rebaudioside N7 in the highly purified composition of rebaudioside N7 that this invention provides is greater than about 80%, greater than about 90%, greater than about 91%, greater than about 92%, greater than about 93%, greater than about 94%, greater than about 95%, greater than about 96%, greater than about 97%, greater than about 98% or greater than about 99% by weight on a dried basis.

The content of rebaudioside N8 in the highly purified composition of rebaudioside N8 that this invention provides is greater than about 80%, greater than about 90%, greater than about 91%, greater than about 92%, greater than about 93%, greater than about 94%, greater than about 95%, greater than about 96%, greater than about 97%, greater than about 98% or greater than about 99% by weight on a dried basis.

This invention also provides a consumable product comprising rebaudioside N7, wherein the product is selected from the group consisting of food, beverages, pharmaceutical compositions, tobacco products, nutraceutical compositions, oral hygiene compositions, and cosmetic compositions.

This invention also provides a consumable product comprising rebaudioside N8, wherein the product is selected from the group consisting of food, beverages, pharmaceutical compositions, tobacco products, nutraceutical compositions, oral hygiene compositions, and cosmetic compositions.

Also, the consumable product that this invention provides is selected from the group consisting of foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, lipsticks, and many others.

The consumable product that this invention provides further comprises at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, flavorings with modifying properties (FMP), astringent compounds, proteins or protein hydrolysates, surfactants, emulsifiers, flavonoids, alcohols, polymers and combinations thereof.

The consumable product that this invention provides further comprises at least one functional ingredient selected from the group consisting of saponins, antioxidants, dietary fiber sources, fatty acids, vitamins, glucosamine, minerals, preservatives, hydration agents, probiotics, prebiotics, postbiotics, weight management agents, osteoporosis management agents, phytoestrogens, long chain primary aliphatic saturated alcohols, phytosterols and combinations thereof.

The consumable product that this invention provides further comprises a compound selected from the group consisting of dulcoside A, dulcoside B, dulcoside C, dulcoside D, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f, rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s, rebaudioside A, rebaudioside A1G, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside C8, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside D14, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside E14, rebaudioside F, rebaudioside F1, rebaudioside F2, rebaudioside F3, rebaudioside F4, rebaudioside F5, rebaudioside G, rebaudioside H, rebaudioside H1, rebaudioside H2, rebaudioside H3, rebaudioside H4, rebaudioside H5, rebaudioside H6, rebaudioside H7, rebaudioside H8, rebaudioside I, rebaudioside I2, rebaudioside I3, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside N8, rebaudioside 0, rebaudioside 02, rebaudioside 03, rebaudioside 04, rebaudioside 05, rebaudioside 06, rebaudioside 07, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside U4, rebaudioside U5, rebaudioside U6, rebaudioside V, rebaudioside V2, rebaudioside V3, rebaudioside VIII, rebaudioside VIIIa, rebaudioside VIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolbioside H, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, stevioside N, SvG7, NSF-02, Mogroside V, siratose, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose, 5-ketofructose and combinations thereof.

This invention also provides a method for enhancing the sweetness of a beverage or food product, comprising a sweetener, comprising providing a beverage or food product comprising a sweetener; and adding a sweetness enhancer comprising rebaudioside N7, wherein rebaudioside N7 is present in a concentration at or below the sweetness recognition threshold or in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for enhancing the sweetness of a beverage or food product, comprising a sweetener, comprising providing a beverage or food product comprising a sweetener; and adding a sweetness enhancer comprising rebaudioside N8, wherein rebaudioside N8 is present in a concentration at or below the sweetness recognition threshold or in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for stabilizing the flavor of a beverage or food product, comprising providing a beverage or food product; and adding a flavor stabilizer comprising rebaudioside N7, wherein rebaudioside N7 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for stabilizing the flavor of a beverage or food product, comprising providing a beverage or food product; and adding a flavor stabilizer comprising rebaudioside N8, wherein rebaudioside N8 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for modification (including enhancing or suppressing) of flavor and/or taste profile of a beverage or food product, comprising providing a beverage or food product; and adding a flavoring with modifying properties (FMP) comprising rebaudioside N7, wherein rebaudioside N7 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight

This invention also provides a method for modification (including enhancing or suppressing) of flavor and/or taste profile of a beverage or food product, comprising providing a beverage or food product; and adding a flavoring with modifying properties (FMP) comprising rebaudioside N8, wherein rebaudioside N8 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight This invention also provides a method for suppressing foaming of a beverage or food product, comprising providing a beverage or food product; and adding a foam suppressor comprising rebaudioside N7, wherein rebaudioside N7 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for suppressing foaming of a beverage or food product, comprising providing a beverage or food product; and adding a foam suppressor comprising rebaudioside N8, wherein rebaudioside N8 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for enhancing the solubility of insoluble material in a beverage or food product, comprising providing a beverage or food product containing insoluble material; and adding a solubility enhancing agent comprising rebaudioside N7, wherein rebaudioside N7 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for enhancing the solubility of insoluble material in a beverage or food product, comprising providing a beverage or food product containing insoluble material; and adding a solubility enhancing agent comprising rebaudioside N8, wherein rebaudioside N8 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

The following examples illustrate preferred embodiments of the invention for the preparation of highly purified target steviol glycoside(s), particularly steviolmonoside, steviolmonoside A, steviolbioside A, steviolbioside C, steviolbioside D, steviolbioside G, rubusoside, stevioside A, stevioside K, stevioside L, stevioside M, dulcoside A, dulcoside C, rebaudioside G, rebaudioside C, rebaudioside C5, rebaudioside C7, rebaudioside C8, rebaudioside E4, rebaudioside E12, rebaudioside E13, rebaudioside E14, rebaudioside D13, rebaudioside D14, rebaudioside H7, rebaudioside H8, rebaudioside K, rebaudioside K2, rebaudioside N7 and/or rebaudioside N8. It will be understood that the invention is not limited to the materials, proportions, conditions and procedures set forth in the examples, which are only illustrative.

### EXAMPLES

### Example 1

### Protein Sequences of Engineered Enzymes Used in the Biocatalytic Process

SEQ ID 1:
>SuSy_At, variant PM1-54-2-E05 (engineered sucrose synthase;
source of artificial gene: Arabidopsis thaliana)
MANAERMITRVHSQRERLNETLVSERNEVLALLSRVEAKGKGILQQNQIIAEFEALPEQT
RKKLEGGPFFDLLKSTQEAIVLPPWVALAVRPRPGVWEYLRVNLHALVVEELQPAEFLHE
KEELVDGVKNGNFTLELDFEPFNASIPRPTLHKYIGNGVDFLNRHLSAKLFHDKESLLPL
LDFLRLHSHQGKNLMLSEKIQNLNTLQHTLRKAEEYLAELKSETLYEEFEAKFEEIGLER
GWGDNAERVLDMIRLLLDLLEAPDPSTLETFLGRVPMVFNVVILSPHGYFAQDNVLGYPD
TGGQVVYILDQVRALEIEMLQRIKQQGLNIKPRILILTRLLPDAVGTTCGERLERVYDSE
YCDILRVPFRTEKGIVRKWISRFEVWPYLETYTEDAAVELSKELNGKPDLIIGNYSDGNL
VASLLAHKLGVTQCTIAHALEKTKYPDSDIYWKKLDDKYHFSCQFTADIFAMNHTDFIIT
STFQEIAGSKETVGQYESHTAFTLPGLYRVVHGIDVFDPKFNIVSPGADMSIYFPYTEEK
RRLTKFHSEIEELLYSDVENDEHLCVLKDKKKPILFTMARLDRVKNLSGLVEWYGKNTRL
RELVNLVVVGGDRRKESKDNEEKAEMKKMYDLIEEYKLNGQFRWISSQMDRVRNGELYRY
ICDTKGAFVQPALYEAFGLTVVEAMTCGLPTFATCKGGPAEIIVHGKSGFHIDPYHGDQA
ADLLADFFTKCKEDPSHWDEISKGGLQRIEEKYTWQIYSQRLLTLTGVYGFWKHVSNLDR
LEHRRYLEMFYALKYRPLAQAVPLAQDD
SEQ ID 2:
>UGT74G1 (glucosyltransferase; source of WT gene: Stevia rebaudiana)
MAEQQKIKKSPHVLLIPFPLQGHINPFIQFGKRLISKGVKTTLVTTIHTLNSTLNHSNTT
TTSIEIQAISDGCDEGGEMSAGESYLETFKQVGSKSLADLIKKLQSEGTTIDAIIYDSMT
EWVLDVAIEFGIDGGSFFTQACVVNSLYYHVHKGLISLPLGETVSVPGFPVLQRWETPLI
LQNHEQIQSPWSQMLFGQFANIDQARWVFTNSFYKLEEEVIEWTRKIWNLKVIGPTLPSM
YLDKRLDDDKDNGFNLYKANHHECMNWLDDKPKESVVYVAFGSLVKHGPEQVEEITRALI
DSDVNFLWVIKHKEEGKLPENLSEVIKTGKGLIVAWCKQLDVLAHESVGCFVTHCGENST
LEAISLGVPVVAMPQFSDQTTNAKLLDEILGVGVRVKADENGIVRRGNLASCIKMIMEEE
RGVIIRKNAVKWKDLAKVAVHEGGSSDNDIVEFVSELIKA
SEQ ID 3:
>UGT85C2 (glucosyltransferase; source of WT gene: Stevia rebaudiana)
MDAMATTEKKPHVIFIPFPAQSHIKAMLKLAQLLHHKGLQITFVNTDFIHNQFLESSGPH
CLDGAPGFRFETIPDGVSHSPEASIPIRESLLRSIETNFLDRFIDLVTKLPDPPTCIISD
GFLSVFTIDAAKKLGIPVMMYWTLAACGFMGFYHIHSLIEKGFAPLKDASYLTNGYLDTV
IDWVPGMEGIRLKDFPLDWSTDLNDKVLMFTTEAPQRSHKVSHHIFHTFDELEPSIIKTL
SLRYNHIYTIGPLQLLLDQIPEEKKQTGITSLHGYSLVKEEPECFQWLQSKEPNSVVYVN
FGSTTVMSLEDMTEFGWGLANSNHYFLWIIRSNLVIGENAVLPPELEEHIKKRGFIASWC
SQEKVLKHPSVGGFLTHCGWGSTIESLSAGVPMICWPYSWDQLTNCRYICKEWEVGLEMG
TKVKRDEVKRLVQELMGEGGHKMRNKAKDWKEKARIAIAPNGSSSLNIDKMVKEITVLAR
N
SEQ ID 4:
>UGTS12 variant 0234 (engineered glucosyltransferase;
source of artificial gene: Solanum lycopersicum)
MATNLRVLMFPWLAYGHISPFLNIAKQLADRGFLIYLCSTRINLESIIKKIPEKYADSIH
LIELQLPELPELPPHYHTTNGLPPHLNPTLHKALKMSKPNFSRILQNLKPDLLIYDVLQP
WAEHVANEQGIPAGKLLVSCAAVFSYFFSFRKNPGVEFPFPAIHLPEVEKVKIREILAKE
PEEGGRLDEGNKQMMLMCTSRTIEAKYIDYCTELCNWKVVPVGPPFQDLITNDADNKELI
DWLGTKPENSTVFVSFGSEYFLSKEDMEEIAFALEASNVNFIWVVRFPKGEERNLEDALP
EGFLERIGERGRVLDKFAPQPRILNHPSTGGFISHCGWNSVMESIDFGVPIIAMPIHNDQ
PINAKLMVELGVAVEIVRDDDGKIHRGEIAEALKSVVTGETGEILRAKVREISKNLKSIR
DEEMDAVAEELIQLCRNSNKSK
SEQ ID 5:
>UGT76G1 variant 0042 (engineered glucosyltransferase;
source of artificial gene: Stevia rebaudiana)
MENKTETTVRRRRRIILFPVPFQGHINPILQLANVLYSKGFAITILHTNFNKPKTSNYPH
FTFRFILDNDPQDERISNLPTHGPLAGMRIPIINEHGADELRRELELLMLASEEDEEVSC
LITDALWYFAQDVADSLNLRRLVLMTSSLFNFHAHVSLPQFDELGYLDPDDKTRLEEQAS
GFPMLKVKDIKSAYSNWQIGKEILGKMIKQTKASSGVIWNSFKELEESELETVIREIPAP
SFLIPLPKHLTASSSSLLDHDRTVFEWLDQQAPSSVLYVSFGSTSEVDEKDFLEIARGLV
DSGQSFLWVVRPGFVKGSTWVEPLPDGFLGERGKIVKWVPQQEVLAHPAIGAFWTHSGWN
STLESVCEGVPMIFSSFGGDQPLNARYMSDVLRVGVYLENGWERGEVVNAIRRVMVDEEG
EYIRQNARVLKQKADVSLMKGGSSYESLESLVSYISSL
SEQ ID 6:
>URhaT12V1 (rhamnosyltransferase; source of WT gene:
Oryza sativa Japonica; SEQ ID 36,507 US 2006/0123505 A1)
MDSGYSSSYAAAAGMHVVICPWLAFGHLLPCLDLAQRLASRGHRVSFVSTPRNISRLPPV
RPALAPLVAFVALPLPRVEGLPDGAESTNDVPHDRPDMVELHRRAFDGLAAPFSEFLGTA
CADWVIVDVFHHWAAAAALEHKVPCAMMLLGSAHMIASIADRRLERAETESPAAAGQGRP
AAAPTFEVARMKLIRTKGSSGMSLAERFSLTLSRSSLVVGRSCVEFEPETVPLLSTLRGK
PITFLGLMPPLHEGRREDGEDATVRWLDAQPAKSVVYVALGSEVPLGVEKVHELALGLEL
AGTRFLWALRKPTGVSDADLLPAGFEERTRGRGVVATRWVPQMSILAHAAVGAFLTHCGW
NSTIEGLMFGHPLIMLPIFGDQGPNARLIEAKNAGLQVARNDGDGSFDREGVAAAIRAVA
VEEESSKVFQAKAKKLQEIVADMACHERYIDGFIQQLRSYKD
SEQ ID 7:
>URhaT22V1 (translocated URhaT12V1; source of artificial
gene: Oryza sativa Japonica; SEQ ID 9 US 2017/0332673 A1)
MAGSSGMSLAERFSLTLSRSSLVVGRSCVEFEPETVPLLSTLRGKPITFLGLMPPLHEGR
REDGEDATVRWLDAQPAKSVVYVALGSEVPLGVEKVHELALGLELAGTRFLWALRKPTGV
SDADLLPAGFEERTRGRGVVATRWVPQMSILAHAAVGAFLTHCGWNSTIEGLMFGHPLIM
LPIFGDQGPNARLIEAKNAGLQVARNDGDGSFDREGVAAAIRAVAVEEESSKVFQAKAKK
LQEIVADMACHERYIDGFIQQLRSYKDDSGYSSSYAAAAGMHVVICPWLAFGHLLPCLDL
AQRLASRGHRVSFVSTPRNISRLPPVRPALAPLVAFVALPLPRVEGLPDGAESTNDVPHD
RPDMVELHRRAFDGLAAPFSEFLGTACADWVIVDVFHHWAAAAALEHKVP CAMMLLGSAH
MIASIADRRLERAETESPAAAGQGRPAAAPTFEVARMKLIRTK
SEQ ID 8:
>URhaT32V1 (rhamnosyltransferase; source of WT gene:
Hordeum vulgare; AK362851 doi: 10.1038/nature11543)
MDGDGNSSSSSSPLHVVICPWLALGHLLPCLDIAERLASRGHRVSFVSTPRNIARLPPLR
PAVAPLVEFVALPLPHVDGLPEGAESTNDVPYDKFELHRKAFDGLAAPFSEFLRAACAEG
AGSRPDWLIVDTFHHWAAAAAVENKVPCVMLLLGAATVIAGFARGVSEHAAAAVGKERPA
AEAPSFETERRKLMTTQNASGMTVAERYFLTLMRSDLVAIRSCAEWEPESVAALTTLAGK
PVVPLGLLPPSPEGGRGVSKEDAAVRWLDAQPAKSVVYVALGSEVPLRAEQVHELALGLE
LSGARFLWALRKPTDAPDAAVLPPGFEERTRGRGLVVTGWVPQIGVLAHGAVAAFLTHCG
WNSTIEGLLFGHPLIMLPISSDQGPNARLMEGRKVGMQVPRDESDGSFRREDVAATVRAV
AVEEDGRRVFTANAKKMQEIVADGACHERCIDGFIQQLRSYKA
SEQ ID 9:
>URhaT42V1 (rhamnosyltransferase; source of WT gene: Stevia
rebaudiana; NCBI Reference Sequence: ACE87855.1)
MATSDSIVDDRKQLHVATFPWLAFGHILPYLQLSKLIAEKGHKVSFLSTTRNIQRLSSHI
SPLINVVQLTLPRVQELPEDAEATTDVHPEDIPYLKKASDGLQPEVTRFLEQHSPDWIIY
DYTHYWLPSIAASLGISRAHFSVTTPWAIAYMGPSADAMINGSDGRTTVEDLTTPPKWFP
FPTKVCWRKHDLARLVPYKAPGISDGYRMGLVLKGSDCLLSKCYHEFGTQWLPLLETLHQ
VPVVPVGLLPPEVPGDEKDETWVSIKKWLDGKQKGSVVYVALGSEVLVSQTEVVELALGL
ELSGLPFVWAYRKPKGPAKSDSVELPDGFVERTRDRGLVWTSWAPQLRILSHESVCGFLT
HCGSGSIVEGLMFGHPLIMLPIFGDQPLNARLLEDKQVGIEIPRNEEDGCLTKESVARSL
RSVVVEKEGEIYKANARELSKIYNDTKVEKEYVSQFVDYLEKNTRAVAIDHES
SEQ ID 10:
>URhaT52V1 (rhamnosyltransferase; source of WT gene:
Oryza brachyantha; XP_006650455.2 doi: 10.1038/ncomms2596)
MENGSSPLHVVIFPWLAFGHLLPFLDLAERLAARGHRVSFVSTPRNLARLRPVRPALRGL
VDLVALPLPRVDGLPDGAEATSDVPFEKFELHRKAFDGLAAPFSAFLDAACAGDKRPDWV
IPDFMHYWVAAAAQKRGVPCAVLIPCSADVMALYGQPTETSTEQPEAIARSMAAEAPSFE
AERNTEEYGTAGASGVSIMTRFSLTLKWSKLVALRSCPELEPGVFTTLTRVYSKPVVPFG
LLPPRRDGAHGVRKNGEDDGAIIRWLDEQPAKSVVYVALGSEAPVSADLLRELAHGLELA
GTRFLWALRRPAGVNDGDSILPNGFLERTGERGLVTTGWVPQVSILAHAAVCAFLTHCGW
GSVVEGLQFGHPLIMLPIIGDQGPNARFLEGRKVGVAVPRNHADGSFDRSGVAGAVRAVA
VEEEGKAFAANARKLQEIVADRERDERCTDGFIHHLTSWNELEA

### Example 2

### Expression and Formulation of SuSy_at Variant of SEQ ID 1

The gene coding for the SuSy_At variant of SEQ ID 1 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.2 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with distilled water, resulting in an enzymatic active preparation.

For enzymatic active preparations of SuSy_At, activity in Units is defined as follows: 1 mU of SuSy_At turns over 1 nmol of sucrose into fructose in 1 minute. Reaction conditions for the assay are 30° C., 50 mM potassium phosphate buffer pH 7.0, 400 mM sucrose at to, 3 mM MgCl₂, and 15 mM uridine diphosphate (UDP).

### Example 3

### Expression and Formulation of UGTS12 Variant of SEQ ID 4

The gene coding for the UGTS12 variant of SEQ ID 4 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGTS12, activity in Units is defined as follows: 1 mU of UGTS12 turns over 1 nmol of rebaudioside A (Reb A) into rebaudioside D (Reb D) in 1 minute. Reaction conditions for the assay are 30° C., 50 mM potassium phosphate buffer pH 7.0, 10 mM Reb A at to, 500 mM sucrose, 3 mM MgCl₂, 0.25 mM uridine diphosphate (UDP) and 3 U/mL of SuSy_At.

### Example 4

### Expression and Formulation of UGT76G1 Variant of SEQ ID 5

The gene coding for the UGT76G1 variant of SEQ ID 5 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGT76G1, activity in Units is defined as follows: 1 mU of UGT76G1 turns over 1 nmol of rebaudioside D (Reb D) into rebaudioside M (Reb M) in 1 minute. Reaction conditions for the assay are 30° C., 50 mM potassium phosphate buffer pH 7.0, 10 mM Reb D at to, 500 mM sucrose, 3 mM MgCl₂, 0.25 mM uridine diphosphate (UDP) and 3 U/mL of SuSy_At.

### Example 5

### Synthesis of Rebaudioside N7 and Rebaudioside N8 in a One-Pot Reaction, Adding UGTS12 and SuSy_at at the Same Time.

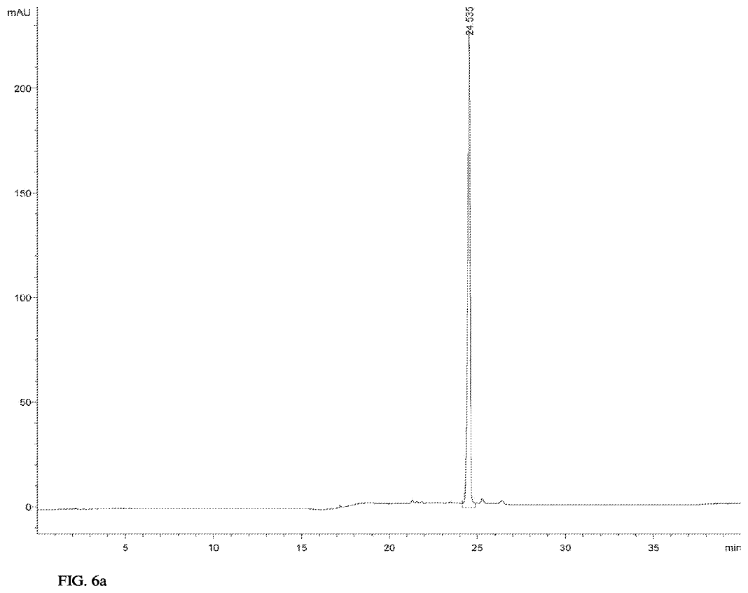

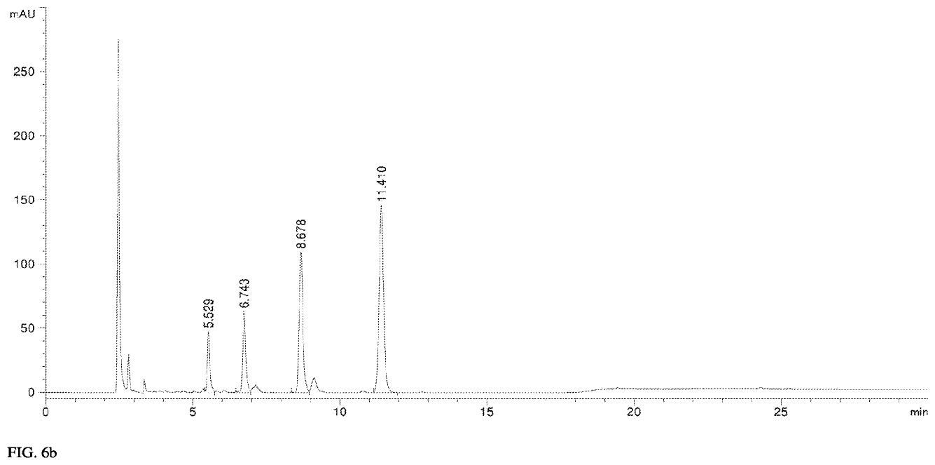

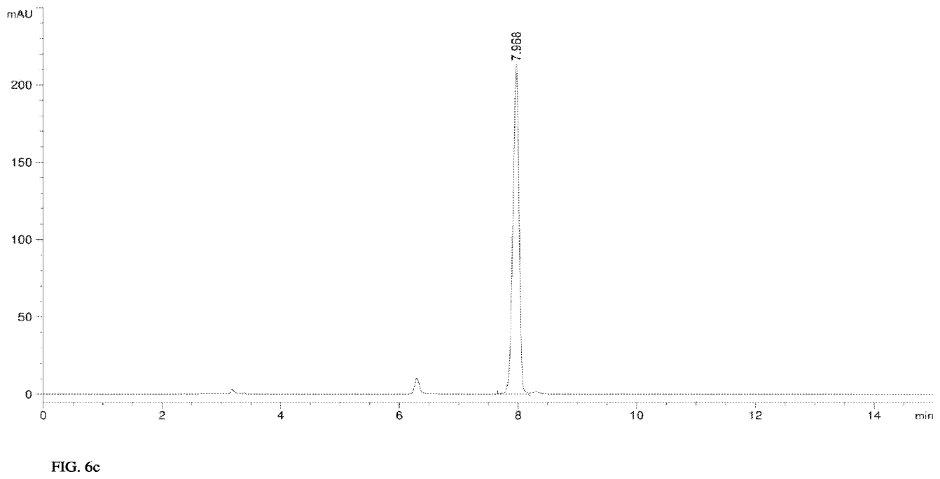

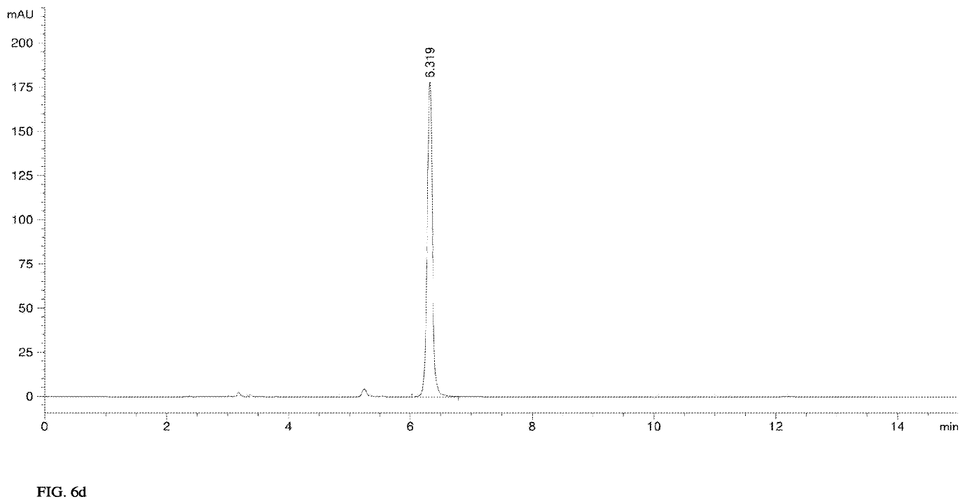

Rebaudioside K, rebaudioside N7, rebaudioside N8 and various steviol glycoside molecules were synthesized directly from rebaudioside C (see FIG. 6a) in a one-pot reaction, utilizing the two enzymes (see EXAMPLES 1, 2 and 3): UGTS12 (variant of SEQ ID 4) and SuSy_At (variant of SEQ ID 1).

The final reaction solution contained approximately 105 U/L UGTS12, 405 U/L SuSy_At, 5 mM rebaudioside C, 0.25 mM uridine diphosphate (UDP), 1 M sucrose, 4 mM MgCl₂ and potassium phosphate buffer (pH 6.6). First, 207 mL of distilled water were mixed with 0.24 g MgCl₂.6H₂O, 103 g sucrose, 9.9 mL of 1.5 M potassium phosphate buffer (pH 6.6) and 15 g rebaudioside C. The final volume of the reaction mixture was adjusted to 300 mL.

After dissolving the components, the temperature was adjusted to 45° C. and UGTS12, SuSy_At and 39 mg UDP were added. The reaction mixture was incubated at 45° C. shaker for 48 hrs. Additional 39 mg UDP was added at 12 hrs, 24 hrs and 36 hrs. The content of reb N7, reb N8, reb K and various steviol glycosides at the end of the reaction (48 hours) was analyzed by HPLC.

### Example 6

### HPLC Analysis

For analysis, biotransformation samples were inactivated by adjusting the pH to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB—C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Steviol glycoside species were detected by UV at 210 nm.

Table 1 shows for each time point the conversion of reb C into identified steviol glycoside species (area percentage). The chromatograms of the starting material rebaudioside C and the reaction mixture at 48 hours are shown in FIG. 6a and FIG. 6b respectively. Those with skill in the art will appreciate that retention times can occasionally vary with changes in solvent and/or equipment.

TABLE 1
Biotransformation of reb C (rt 24.535 min) to reb
K (11.410 min), reb N7 (8.678 min), reb N8 (6.743
min) and other steviol glycoside species

% conversion from rebaudioside C

| Peak | reaction time 0 hr | reaction time 48 hr |
|---|---|---|
| rt 5.529 | 0 | 6.27 |
| rt 6.743 | 0 | 11.66 |
| rt 8.678 | 0 | 24.90 |
| rt 11.410 | 0 | 57.17 |
| rt 24.535 | 100 | 0 |

### Example 7

### Purification of Rebaudioside N7 and Rebaudioside N8

300 mL of the reaction mixture of EXAMPLE 5, (after 48 hrs), was inactivated by adjusting the pH to pH 5.5 with H₃PO₄ and then boiled for 10 minutes and filtered using cardboard with pore size of 3 μm. The filtrate was loaded into a column containing 500 mL YWD03 (Cangzhou Yuanwei, China) resin pre-equilibrated with water. The resin was washed with 2.5 L water and the water effluent from this step was discarded.

The steviol glycosides were eluted from the YWD03 resin column with 2.5 L 70% v/v ethanol/water. The effluent from this step was collected and subjected to evaporation on a rotary evaporator. This concentrated sample was subjected to further fractionation and separation by preparative HPLC, using the conditions listed in Table 2 below.

Preparative HPLC fractions that corresponded to individual compounds from multiple runs were combined according to retention time. The samples were freeze-dried to give 15 mg rebaudioside N7 and 11 mg rebaudioside N8 separately.

TABLE 2
Conditions for preparative HPLC

| | |
|---|---|
| Column | Agilent Poroshell 120 SB-C18, 4.6 mm × 150 mm, |
| | 2.7 μm |
| Temperature | 40° C. |
| Mobile Phase | Isocratic - Water 75% Acetonitrile 25% |
| Flow rate | 0.5 mL/min |
| Injection | 10 μL |
| Stop time | 20 mins |
| Autosampler | Ambient |
| temperature | |
| Detection | UV at 210 nm |

The purity of obtained rebaudioside N7 and rebaudioside N8 fraction was evaluated by LCMS method described in EXAMPLE 8. The chromatogram of purified rebaudioside N7 is shown in FIG. 6c. The chromatogram of purified rebaudioside N8 is shown in FIG. 6d. Those with skill in the art will appreciate that retention times can occasionally vary with changes in solvent and/or equipment.

### Example 8

### Structure Elucidation of Rebaudioside N7

NMR experiments were performed on a Bruker 800 MHz spectrometer, with the rebaudioside N7 sample, prepared according to EXAMPLE 7, dissolved in pyridine-d5. Along with signals from the sample, signals from pyridine-d5 at Sc 123.5, 135.5, 149.9 ppm and SH 7.19, 7.55, 8.71 ppm were observed. 1H-NMR and APT NMR spectra of rebaudioside N7 confirmed the excellent quality of the sample. HSQC shows the presence of an exo-methylene group in the sugar region, with a long-range coupling to C-15, observable in the H,H-COSY. Correlation of the signals in the HSQC, HMBC and H,H-COSY reveal the presence of steviol glycoside with the following aglycone structure:

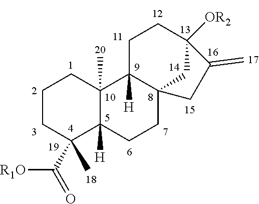

Correlation of HSQC and HMBC shows the presence six anomeric signals, marked as 1a, 1b, 1c, 1d, 1e and 1f. Sugar A was assigned as α-L-rhamnopyranoside based on coupling constants of H6a, H5a and NOESY correlations. For sugars B through E, the coupling constant of the anomeric protons of about 8 Hz and/or the NOE-correlations of the anomeric protons allow the identification of these five sugars as β-D-glucopyranosides.

Combined data from HSQC and HMBC reveal the sugar-sugar linkages and sugar-aglycone linkages. The assignment of the sugar sequence was further supported by TOCSY, H2BC.

Altogether, results from NMR experiments above were used to assign the chemical shifts of the protons and carbons of the structure of rebaudioside N7 (see Table 3).

TABLE 3
Chemical shifts of rebaudioside N7

| | | | | HMBC | H2BC |
|---|---|---|---|---|---|
| Position | δ_{C} [ppm] | δ_{H} [ppm] | Int | (H→C) | (H→C) |

Aglycone moiety

| | | | | | | | |
|---|---|---|---|---|---|---|---|
| 1 | 40.50 | CH2 | 1.66 | m | | 10 | |
| | | | 0.69 | m | | | 2 |
| 2 | 19.95 | CH2 | 2.09 | m | | | |
| | | | 1.42 | m | | | |
| 3 | 37.68 | CH2 | 2.78 | m | | | |
| | | | 1.08 | m | | 4 | 2 |
| 4 | 44.35 | C | — | — | | | |
| 5 | 57.33 | CH | 0.96 | m | | 4, 10, 18, | 6 |
| | | | | | | 19, 20 | |
| 6 | 22.03 | CH2 | 2.18 | m | | | 5, 7 |
| | | | 1.88 | m | | 8 | |
| 7 | 41.63 | CH2 | 1.40 | m | | 8 | |
| | | | 1.25 | m | | 8 | 6 |
| 8 | 42.50 | C | — | — | | | |
| 9 | 53.81 | CH | 0.85 | m | | 1, 8, 10, 12, | 11 |
| | | | | | | 14, 15, 20 | |
| 10 | 39.61 | C | — | — | | | |
| 11 | 20.61 | CH2 | 1.63 | m | 2H | 8 | 12 |
| 12 | 38.17 | CH2 | 2.13 | m | | 13, 16 | 11 |
| | | | 2.03 | m | | | |
| 13 | 86.57 | C | — | — | | | |
| 14 | 43.11 | CH2 | 2.39 | m | | 13, 16 | |
| | | | 1.97 | m | | 8, 13 | |
| 15 | 48.14 | CH2 | 2.11 | m | | 8, 13, 16, 17 | |
| | | | 2.00 | m | | 8 | |
| 16 | 154.27 | C | — | — | | | |
| 17 | 104.83 | CH2 | 5.63 | m | | 13, 16 | |
| | | | 5.06 | m | | 13, 16 | |
| 18 | 29.29 | CH3 | 1.41 | s | 3H | 4, 19 | |
| 19 | 175.76 | C | — | — | | | |
| 20 | 16.69 | CH3 | 1.06 | s | 3H | | |

| | | | | H2BC | HMBC | | |
|---|---|---|---|---|---|---|---|
| Position | δ_{C} [ppm] | δ_{H} [ppm] | J [Hz] | (H→C) | (H→C) | NOESY | COSY |

Sugar A: α-L-Rhamnopyranoside

| | | | | | | | | |
|---|---|---|---|---|---|---|---|---|
| 1a | 101.51 | CH | 6.51 | brs | | 2e | | 2a |
| 2a | 72.45 | CH | 4.80 | | | | 3a | |
| 3a | 72.40 | CH | 4.57 | | | | 2a, 5a | |
| 4a | 74.05 | CH | 4.28 | | | | | |
| 5a | 69.42 | CH | 4.88 | dq | 9.2, 6.2 | | 3a | |
| 6a | 18.96 | CH3 | 1.7 | d | 6.2 | | | |

Sugar B: β-D-Glucopyranoside

| | | | | | | | | | |
|---|---|---|---|---|---|---|---|---|---|
| 1b | 93.51 | CH | 6.28 | d | 7.7 | 2b | 19 | 3b, 5b | 2b |
| 2b | 81.17 | CH | 4.38 | m | | | | | |
| 3b | 78.20 | CH | 4.30 | m | | | | | |
| 4b | 70.56 | CH | 4.27 | m | | | | | |
| 5b | 79.16 | CH | 3.90 | m | | | | | |
| 6b | 62.01 or | CH2 | 4.37 | m | | | | | |
| | 62.05 | | 4.29 | m | | | | | |

Sugar C: β-D-Glucopyranoside

| | | | | | | | | | |
|---|---|---|---|---|---|---|---|---|---|
| 1c | 105.66 | CH | 5.44 | d | 7.7 | 2c | 2b | 3c, 5c | 2c |
| 2c | 76.29 | CH | 4.02 | m | | | | | |
| 3c | 78.05 | CH | 4.23 | m | | | | | |
| 4c | 71.77 | CH | 4.24 | m | | | | | |
| 5c | 78.53 | CH | 3.97 | m | | | | | |
| 6c | 62.90 | CH2 | 4.55 | m | | | | | |
| | | | 4.44 | m | | | | | |

Sugar D: β-D-Glucopyranoside

| | | | | | | | | | |
|---|---|---|---|---|---|---|---|---|---|
| 1d | 104.96 | CH | 5.00 | d | 7.9 | 2d | 3e, 3d, | 3d, 5d | 2d |
| | | | | | | | 5d | | |
| 2d | 74.58 | CH | 3.97 | m | | | | | |
| 3d | 78.39 | CH | 4.14 | m | | | | | |
| 4d | 71.91 | CH | 3.91 | m | | | | | |
| 5d | 76.40 | CH | 4.22 | m | | | | | 6d |
| 6d | 70.12 | CH2 | 5.02 | m | | | | | 5d |
| | | | 3.84 | m | | | | | 5d |

Sugar E: β-D-Glucopyranoside

| | | | | | | | | | |
|---|---|---|---|---|---|---|---|---|---|
| 1e | 98.05 | CH | 4.91 | d | 7.9 | 2e | 13 | 3e, 5e | 2e |
| 2e | 75.69 | CH | 4.21 | m | | 3e | | | 1e, 3e |
| 3e | 91.46 | CH | 3.98 | m | | 4e | | | 2e |
| 4e | 69.91 | CH | 4.00 | m | | 3e, 5e | | | |
| 5e | 76.84 | CH | 3.55 | m | | | | | |
| 6e | 62.05 or | CH2 | 4.29 | m | | | | | |
| | 62.01 | | 4.12 | m | | | | | |

Sugar F: β-D-Glucopyranoside

| | | | | | | | | | |
|---|---|---|---|---|---|---|---|---|---|
| 1f | 104.39 | CH | 4.78 | d | 7.8 | 2f | 6d | 3f, 5f | 2f |
| 2f | 75.56 | CH | 4.09 | m | | | | | |
| 3f | 77.84 | CH | 4.23 | m | | | | | |
| 4f | 71.20 | CH | 4.31 | m | | | | | |
| 5f | 78.53 | CH | 3.90 | m | | | | | |
| 6f | 62.37 | CH2 | 4.49 | m | | | | | |
| | | | 4.37 | m | | | | | |

Correlation of all NMR results indicates rebaudioside N7 with five β-D-glucopyranosides and one α-L-rhamnopyranoside attached to steviol aglycone, as depicted with the following chemical structure:

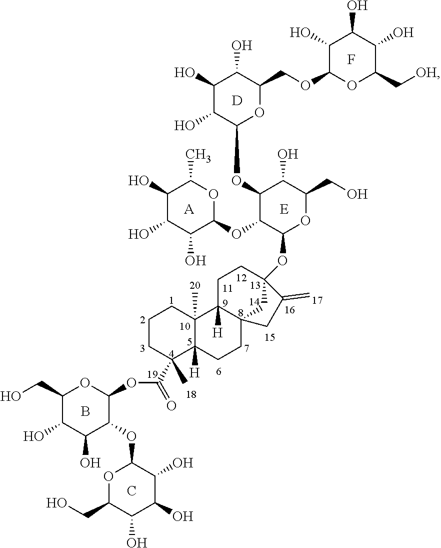

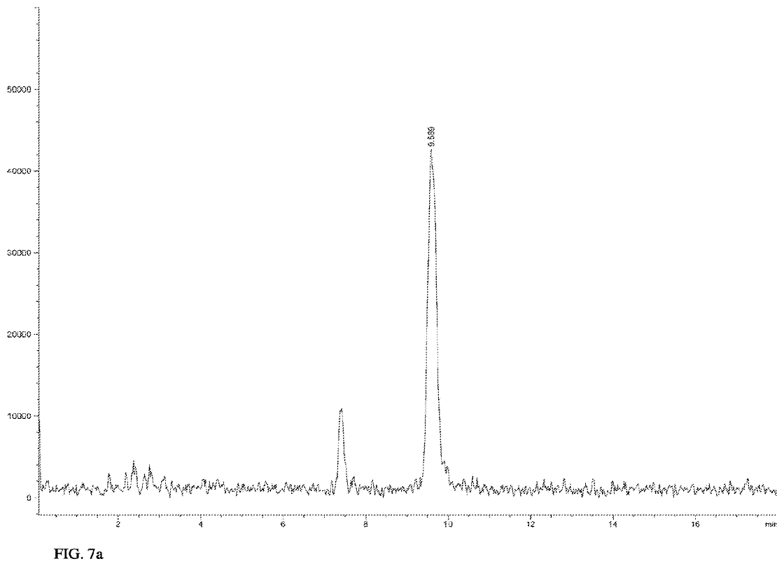

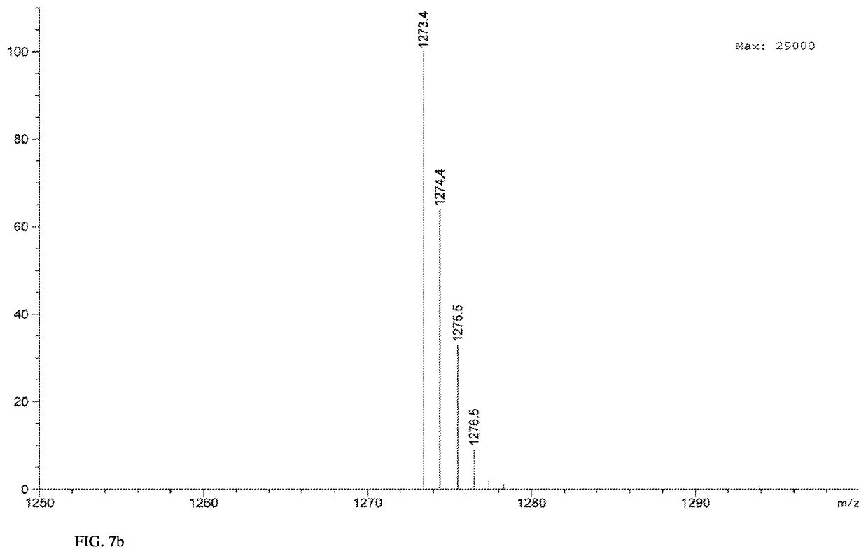

LCMS (FIG. 7a and FIG. 7b) analysis of rebaudioside N7 showed a [M−H]⁻ ion at m/z 1273.4, in good agreement with the expected molecular formula of C56H90032 (calculated for [C₅₆H₈₉O₃₂]⁻ monoisotopic ion: 1273.5). The MS data confirms that rebaudioside N7 has a molecular formula of C₅₆H₉₀O₃₂. LCMS analysis was performed in the following conditions listed in Table 4.

TABLE 4
Conditions for LCMS analysis

| Column | Agilent Poroshell 120 SB-C18, 4.6 mm × | | |
|---|---|---|---|
| | 150 mm, 2.7 μm | | |
| Temperature | 40° C. | | |
| Mobile Phase | A: Mobile Phase Premix Solution | | |
| | 25% Acetonitrile: 75% Formic Acid (0.1% in Water) | | |
| | B: Mobile Phase Premix Solution | | |
| | 32% Acetonitrile: 68% Formic Acid (0.1% in Water) | | |
| Gradient | Time (min) | A (%) | B (%) |
| | 0 | 100 | 0 |
| | 12.0 | 100 | 0 |
| | 12.5 | 50 | 50 |
| | 13.0 | 0 | 100 |
| | 60.0 | 0 | 100 |

| | |
|---|---|
| Flow rate | 0.5 mL/min |
| Injection | 2 μL |
| Run time | 45 mins |
| Post time | 5 mins |
| Autosampler | Ambient |
| temperature | |
| Detection | DAD and MSD at Negative Scan mode |
| DAD Settings | UV at 210 nm (4 nm bandwidth), Reference: 360 nm |
| | (100 nm bandwidth) |
| MSD Setting | Mode: ES-API, Negative Polarity |
| | Drying gas flow: 13.0 L/min |
| | Nebulizer Pressure: 30 psig |
| | Drying gas temperature: 270° C. |
| | Fragmentor: 50 V |
| | Scan ranges: 500 to 1500 of mass |
| Sample | 1 mg/ml (30% ACN in water) |
| Preparation | |

### Example 9

### Structure Elucidation of Rebaudioside N8

NMR experiments were performed on a Bruker 800 MHz spectrometer, with the rebaudioside N8 sample, prepared according to EXAMPLE 7, dissolved in pyridine-d5. Along with signals from the sample, signals from pyridine-d5 at δ_{C} 123.59, 135.63, 149.97 ppm and Sx 7.18, 7.55, 8.69 ppm were observed. ¹H-NMR and APT NMR spectra of rebaudioside N8 confirmed the excellent quality of the sample. HSQC shows the presence of an exo-methylene group in the sugar region. Correlation of the signals in the HSQC, HMBC, H₂BC and H,H—COSY reveal the presence of steviol glycoside with the following aglycone structure:

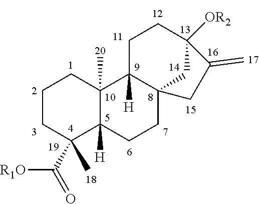

Correlation of HSQC and HMBC shows the presence six anomeric signals, marked as 1a, 1b, 1c, 1d, 1e and 1f. Sugar A was assigned as α-L-rhamnopyranoside based on coupling constants of H6a, H5a and HMBC, HSQC, H2BC correlations. For sugars B through F, the coupling constant of the anomeric protons of about 7.7 Hz and/or the NOE-correlations of the anomeric protons allow the identification of these five sugars as β-D-glucopyranosides.

Combined data from COSY, HSQC and HMBC reveal the sugar-sugar linkages and sugar-aglycone linkages. The assignment of the sugar sequence was further supported by NOESY, TOCSY, H2BC.

Altogether, results from NMR experiments above were used to assign the chemical shifts of the protons and carbons of the structure of rebaudioside N8 (see Table 5).

TABLE 5
Chemical shifts of rebaudioside N8

| | | | | HMBC | H2BC |
|---|---|---|---|---|---|
| Position | δ_{C} [ppm] | δ_{H} [ppm] | Int | (H→C) | (H→C) |

Aglycone moiety

| | | | | | | | |
|---|---|---|---|---|---|---|---|
| 1 | 40.24 | CH2 | 1.68 | m | | 10, 20 | |
| | | | 0.67 | m | | | 2 |
| 2 | 19.78 | CH2 | 2.11 | m | | | |
| | | | 1.43 | m | | | |
| 3 | 37.52 | CH2 | 2.76 | m | | | |
| | | | 1.06 | m | | 4 | |
| 4 | 44.16 | C | — | — | | | |
| 5 | 57.17 | CH | 0.93 | m | | 4 | 6 |
| 6 | 21.78 | CH2 | 2.11 | m | | 8 | 5, 7 |
| | | | 1.82 | m | | 8 | |
| 7 | 41.48 | CH2 | 1.35 | m | | 8 | |
| | | | 1.21 | m | | 8 | 6 |
| 8 | 42.36 | C | — | — | | 11, 14, 15 | |
| 9 | 53.7 | CH | 0.83 | m | | 10, 12, 15, 20 | 11 |
| 10 | 39.46 | C | — | — | | | |
| 11 | 20.43 | CH2 | 1.61 | m | 2H | 8 | 12 |
| 12 | 37.87 | CH2 | 2.03 | m | | 13, 16 | 11 |
| | | | 2.22 | m | | | |
| 13 | 86.25 | C | — | — | | | |
| 14 | 42.79 | CH2 | 2.4 | m | | 13, 16 | |
| | | | 1.96 | m | | 8, 13 | |
| 15 | 47.88 | CH2 | 1.96 | m | | 7, 8, 9, 13, | |
| | | | | | | 14, 16, 17 | |
| | | | 2.09 | m | | 8 | |
| 16 | 154.43 | C | — | — | | | |
| 17 | 104.41 | CH2 | 5.62 | m | | 13, 15, 16 | |
| | | | 5.03 | m | | 13, 15, 16 | |
| 18 | 29.1 | CH3 | 1.39 | s | 3H | 3, 4, 5, 19 | |
| 19 | 175.82 | C | — | — | | | |
| 20 | 16.62 | CH3 | 1.09 | s | 3H | 1, 5, 9, 10 | |

| | | | | H2BC | HMBC | | |
|---|---|---|---|---|---|---|---|
| Position | δ_{C} [ppm] | δ_{H} [ppm] | J [Hz] | (H→C) | (H→C) | NOESY | COSY |

Sugar A: α-L-Rhamnopyranoside

| | | | | | | | | | |
|---|---|---|---|---|---|---|---|---|---|
| 1a | 101.49 | CH | 6.54 | brs | | | 2e | 6a | |
| 2a | 72.25 | CH | 4.88 | | | | | 3a | 1a |
| 3a | 72.20 | CH | 4.61 | | | 4a | | 2a, 5a | |
| 4a | 73.78 | CH | 4.31 | | | 5a | | 6a | 5a |
| 5a | 69.42 | CH | 4.91 | d | 7.79 | 4a | | 6a | 4a |
| 6a | 18.76 | CH3 | 1.72 | d | 5.78 | 5a | | | 5a |

Sugar B: β-D-Glucopyranoside

| | | | | | | | | |
|---|---|---|---|---|---|---|---|---|
| 1b | 93.07 | CH | 6.21 | d | 7.71 | 2b | 19 | 2b |
| 2b | 80.45 | CH | 4.30 | m | | 1b | | 1b, 3b |
| 3b | 77.82 | CH | 4.23 | m | | 2b | | 2b, 4b |
| 4b | 70.30 | CH | 4.23 | m | | 5b | | 3b, 5b |
| 5b | 77.60 | CH | 3.97 | m | | 6b | | 4b, 6b |
| 6b | 69.30 | CH2 | 4.23 | m | | | | 5b |
| | | | 4.64 | m | | 5b | | 5b |

Sugar C: β-D-Glucopyranoside

| | | | | | | | | |
|---|---|---|---|---|---|---|---|---|
| 1c | 105.29 | CH | 5.40 | d | 7.63 | | 2b | 2c |
| 2c | 76.00 | CH | 4.01 | m | | 1c, 3c | 3c | |
| 3c | 77.88 | CH | 4.22 | m | | 4c | 4c | 2c, 4c |
| 4c | 71.60 | CH | 4.22 | m | | | | 5c |
| 5c | 77.49 | CH | 3.97 | m | | | | 4c, 6c |
| 6c | 62.50 | CH2 | 4.56 | m | | | 4c | |
| | | | 4.44 | m | | | | |

Sugar D: β-D-Glucopyranoside

| | | | | | | | | |
|---|---|---|---|---|---|---|---|---|
| 1d | 104.00 | CH | 5.20 | d | 7.68 | | 3e | 2d |
| 2d | 74.87 | CH | 4.00 | m | | 1d, 3d | | 1d, 3d |
| 3d | 78.17 | CH | 4.15 | m | | | | 4d |
| 4d | 71.40 | CH | 4.06 | m | | | | |
| 5d | 78.40 | CH | 4.06 | m | | | | 6d |
| 6d | 62.10 | CH2 | 4.22 | m | | | | |
| | | | 4.56 | m | | | | |

Sugar E: β-D-Glucopyranoside

| | | | | | | | | |
|---|---|---|---|---|---|---|---|---|
| 1e | 98.00 | CH | 5.07 | d | 7.79 | 2e | 13, 2e, | |
| | | | | | | | 3e | |
| 2e | 75.90 | CH | 4.30 | m | | 3e | 1e | 3e |
| 3e | 89.50 | CH | 4.37 | m | | | | 4e |
| 4e | 69.41 | CH | 4.06 | m | | 5e | 3e | |
| 5e | 76.79 | CH | 3.72 | m | | | | 4e, 6e |
| 6e | 62.04 | CH2 | 4.25 | m | | | | |
| | | | 4.12 | m | | | | |

Sugar F: β-D-Glucopyranoside

| | | | | | | | | | |
|---|---|---|---|---|---|---|---|---|---|
| 1f | 105.00 | CH | 4.92 | d | 7.70 | | 6b | 3f, 5f | 2f |
| 2f | 74.80 | CH | 4.09 | m | | 1f | | | |
| 3f | 78.00 | CH | 4.13 | m | | 2f | | | |
| 4f | 71.20 | CH | 4.13 | m | | | | | 5f |
| 5f | 78.10 | CH | 3.80 | m | | 6f | | | |
| 6f | 62.37 | CH2 | 4.46 | m | | | | | 5f |
| | | | 4.31 | m | | | | | |

Correlation of all NMR results indicates rebaudioside N8 with five β-D-glucopyranosides and one α-L-rhamnopyranoside attached to steviol aglycone, as depicted with the following chemical structure:

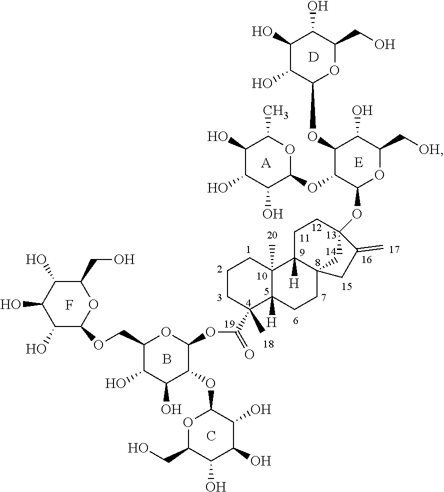

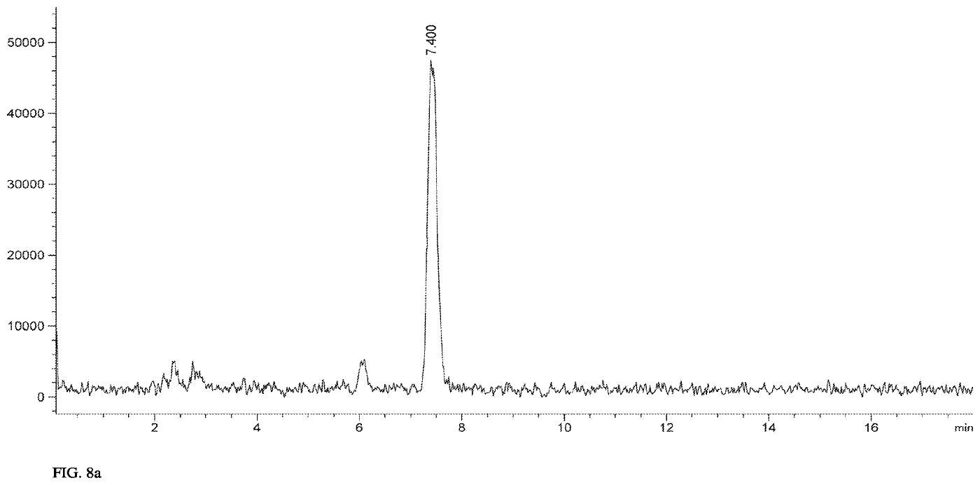

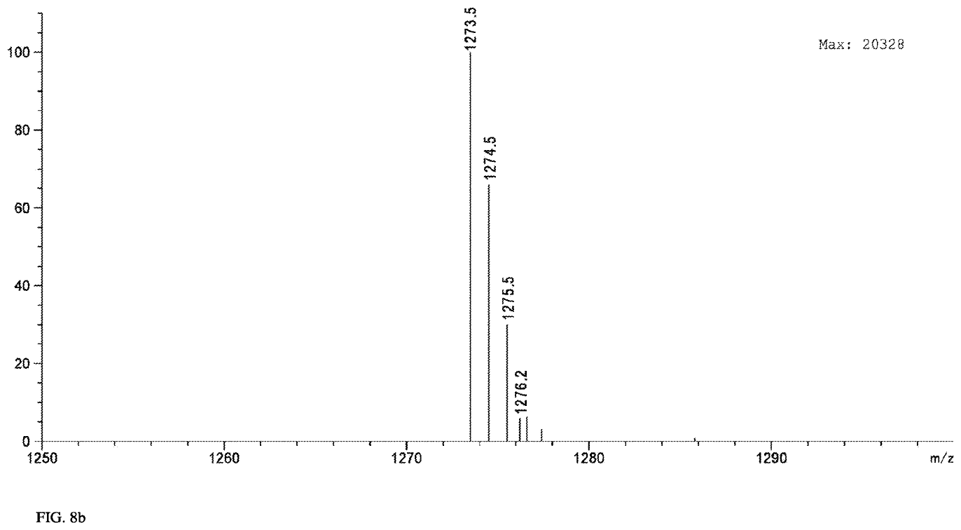

LCMS (FIG. 8a and FIG. 8b) analysis of rebaudioside N8 showed a [M−H]⁻ ion at m/z 1273.5, in good agreement with the expected molecular formula of C56H90032 (calculated for [C₅₆H₈₉O₃₂]⁻ monoisotopic ion: 1273.5). The MS data confirms that rebaudioside N8 has a molecular formula of C₅₆H₉₀O₃₂. LCMS analysis was performed in the conditions listed in Table 4.

We claim:

1. Rebaudioside N7 comprising the following formula:

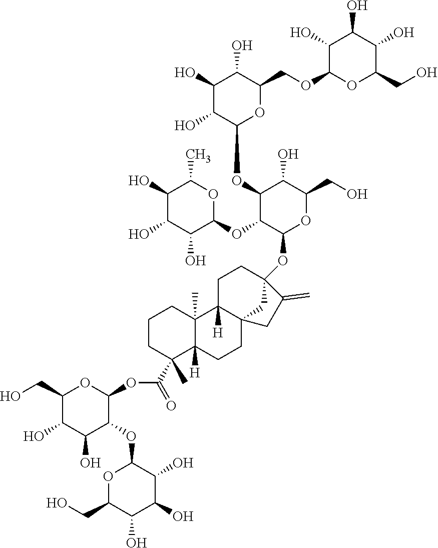

2. Rebaudioside N8 comprising the following formula:

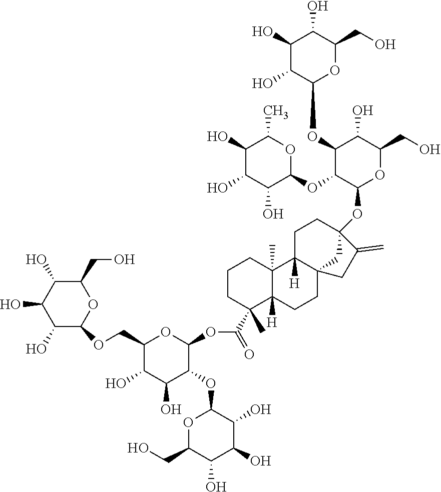

3. A method for producing rebaudioside N7 of claim 1, comprising the steps of:
a. providing a starting composition comprising an organic compound with at least one carbon atom;
b. providing an enzyme preparation or microorganism containing at least one enzyme selected from the group consisting of steviol biosynthesis enzymes, NDP-glucosyltransferases and NDP-rhamnosyltransferases and optionally NDP recycling enzymes;
c. contacting the enzyme preparation or microorganism with a medium containing the starting composition to produce a medium comprising rebaudioside N7.

4. A method for producing rebaudioside N8 of claim 2, comprising the steps of:
a. providing a starting composition comprising an organic compound with at least one carbon atom;
b. providing an enzyme preparation or microorganism containing at least one enzyme selected from the group consisting of steviol biosynthesis enzymes, NDP-glucosyltransferases and NDP-rhamnosyltransferases and optionally NDP recycling enzymes;
c. contacting the enzyme preparation or microorganism with a medium containing the starting composition to produce a medium comprising rebaudioside N8.

5. A method for producing rebaudioside N7 of claim 1, comprising the steps of:
a. providing a starting composition comprising an organic compound with at least one carbon atom;
b. providing a biocatalyst comprising at least one enzyme selected from the group consisting of steviol biosynthesis enzymes, NDP-glucosyltransferases and NDP-rhamnosyltransferases and optionally NDP recycling enzymes;
c. contacting the biocatalyst with a medium containing the starting composition to produce a medium comprising rebaudioside N7.

6. A method for producing rebaudioside N8 of claim 2, comprising the steps of:
a. providing a starting composition comprising an organic compound with at least one carbon atom;
b. providing a biocatalyst comprising at least one enzyme selected from the group consisting of steviol biosynthesis enzymes, NDP-glucosyltransferases and NDP-rhamnosyltransferases and optionally NDP recycling enzymes;
c. contacting the biocatalyst with a medium containing the starting composition to produce a medium comprising rebaudioside N8.

7. The method of claim 3 further comprising the step of:
d. separating rebaudioside N7 from the medium to provide a highly purified composition of rebaudioside N7.

8. (canceled)

9. The method of claim 3, wherein the starting composition is selected from the group consisting of steviol, steviolmonoside, steviolmonoside A, steviolbioside A, steviolbioside C, steviolbioside D, rubusoside, stevioside A, stevioside M, dulcoside A, dulcoside C, rebaudioside G, rebaudioside C, rebaudioside C5, rebaudioside C7, rebaudioside E4, rebaudioside E13, rebaudioside D13, rebaudioside H7, rebaudioside K, other steviol glycosides, polyols, carbohydrates, and combinations thereof.

10. (canceled)

11. The method of claim 3, wherein the microorganism is selected from the group consisting of E. coli, Saccharomyces sp., Aspergillus sp., Pichia sp., Bacillus sp., and Yarrowia sp.

12. The method of claim 5, wherein the biocatalyst is an enzyme, or a cell comprising one or more enzyme, capable of converting the starting composition to rebaudioside N7.

13. The method of claim 6, wherein the biocatalyst is an enzyme, or a cell comprising one or more enzyme, capable of converting the starting composition to rebaudioside N8.

14. The method of claim 3, wherein the enzyme is selected from the group consisting of a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGTS12, UGT76G1, URhaT12V1, URhaT22V1, URhaT32V1, URhaT42V1 or URhaT52V1 or mutant variant thereof having >85% amino-acid sequence identity with SEQ ID 2, SEQ ID 3, SEQ ID 4, SEQ ID 5, SEQ ID 6, SEQ ID 7, SEQ ID 8, SEQ ID 9 and SEQ ID 10, respectively.

15. The method of claim 7, wherein the content of rebaudioside N7 in the highly purified composition of rebaudioside N7 is greater than about 80% by weight on a dried basis.

16. (canceled)

17. A consumable product comprising rebaudioside N7 of claim 1, wherein the product is selected from the group consisting of food, beverages, pharmaceutical compositions, tobacco products, nutraceutical compositions, oral hygiene compositions, and cosmetic compositions.

18. (canceled)

19. The consumable product of claim 17, wherein the product is selected from the group consisting of foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, and lipsticks.

20. The consumable product of claim 17, further comprising at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, flavorings with modifying properties (FMP), astringent compounds, proteins or protein hydrolysates, surfactants, emulsifiers, flavonoids, alcohols, polymers and combinations thereof.

21. The consumable product of claim 17, further comprising at least one functional ingredient selected from the group consisting of saponins, antioxidants, dietary fiber sources, fatty acids, vitamins, glucosamine, minerals, preservatives, hydration agents, probiotics, prebiotics, postbiotics, weight management agents, osteoporosis management agents, phytoestrogens, long chain primary aliphatic saturated alcohols, phytosterols and combinations thereof.

22. The consumable product of claim 17, further comprising a compound selected from the group consisting of dulcoside A, dulcoside B, dulcoside C, dulcosideD, rebaudioside 1a, rebaudioside 1b, rebaudioside 1c, rebaudioside 1d, rebaudioside 1e, rebaudioside 1f rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s, rebaudioside A, rebaudioside AIG, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside C8, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside D14, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside E14, rebaudioside F, rebaudioside F1, rebaudioside F2, rebaudioside F3, rebaudioside F4, rebaudioside F5, rebaudioside G, rebaudioside H, rebaudioside H1, rebaudioside H2, rebaudioside H3, rebaudioside H4, rebaudioside H5, rebaudioside H6, rebaudioside H7, rebaudioside H8, rebaudioside I, rebaudioside I2, rebaudioside I3, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside N8, rebaudioside O, rebaudioside O2, rebaudioside O3, rebaudioside O4, rebaudioside O5, rebaudioside O6, rebaudioside O7, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside U4, rebaudioside U5, rebaudioside U6, rebaudioside V, rebaudioside V2, rebaudioside V3, rebaudioside VIII, rebaudioside VIIIa, rebaudioside VIIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolbioside H, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, stevioside N, SvG7, NSF-02, Mogroside V, siratose, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose, 5-ketofructose and combinations thereof.

23. A method for enhancing the sweetness of a beverage or food product, comprising a sweetener comprising the step of:
a. adding a sweetness enhancer comprising rebaudioside N7 of claim 1 to a beverage or food product comprising a sweetener, wherein rebaudioside N7 is present in a concentration at or below the sweetness recognition threshold.

24. (canceled)

25. The method of claim 23 further comprising the step of adding a sweetness enhancer comprising rebaudioside N7, wherein rebaudioside N7 is present in an amount from about 0.0001% to about 12% by weight.

26. (canceled)

27. A method for stabilizing the flavor of a beverage or food product, comprising the step of:
a. adding a flavor stabilizer comprising rebaudioside N7 of claim 1 to a beverage or food product, wherein rebaudioside N7 is present in an amount from about 0.0001% to about 12% by weight.

28. (canceled)

29. A method for modification of flavor and/or taste profile of a beverage or food product, comprising the step of:
a. adding a flavoring with modifying properties (FMP) comprising rebaudioside N7 of claim 1 to a beverage or food product, wherein rebaudioside N7 is present in an amount from about 0.0001% to about 12% by weight.

30. (canceled)

31. A method for suppressing foaming of a beverage or food product, comprising the step of:
a. adding a foam suppressor comprising rebaudioside N7 of claim 1 to a beverage or food product, wherein rebaudioside N7 is present in an amount from about 0.0001% to about 12% by weight.

32. (canceled)

33. A method for enhancing the solubility of insoluble material in a beverage or food product, comprising the step of:
a. adding a solubility enhancing agent comprising rebaudioside N7 of claim 1 to a beverage or food product containing insoluble material, wherein rebaudioside N7 is present in an amount from about 0.0001% to about 12% by weight.

34. (canceled)
